(19) United States
(12) Patent Application Publication
(10) Pub. No.: US 2026/0228464 A1
(43) Pub. Date: Aug. 6, 2026

(54) Systems and Methods for Automatic Processing of Files for Translation

(71) Applicant: Big Language Solutions, LLC, Atlanta, GA (US)

(72) Inventors: Dan Nelson, Woodland, WA (US); Szilvia Szalai, Cambridge (GB); Yin Fung Khong, Seattle, WA (US)

(73) Assignee: Big Language Solutions, LLC, Atlanta, GA (US)

(21) Appl. No.: 19/045,585

(22) Filed: Feb. 5, 2025

(51) Int. Cl. G06F 40/58; G16H 10/60

(52) U.S. Cl. CPC G06F 40/58; G16H 10/60

(57) ABSTRACT

According to various embodiments, systems and methods are provided for automated processing of a large number of files. In various embodiments, the automatic processing of files supports automatic translation of large volumes of files. In various embodiments, the translation may be based on an input supporting dataset received directly or indirectly from a customer, where the input supporting dataset defines at least one of attribute for the translation. In various embodiments, the automatic processing of files includes a comparison of an original file with one or more templates, and identifying at least one portion of the original file that is different compared to the template. In various embodiments, the translation of files may be performed with the assistance of AI technology.

### CROSS-REFERENCE

None.

### BACKGROUND

The advancement of artificial intelligent (AI) technology is facilitating higher quality and faster translations or processing of documents across a number of languages. Nevertheless, despite the fact that the translation or processing of individual documents can be expedited and improved by such AI technology, the efficient, accurate and fast translation of high numbers of documents has remained a challenge for the industry. In connection with large-scale automatic translations processes and for other applications, there is a particular need for automatic pre-processing of files, and this need is not currently met adequately in the industry.

Consequently, there is a significant need in the industry for improved systems and methods for automatically pre-processing high numbers of files in an automated and scalable manner.

### SUMMARY

Various example embodiments describe systems, methods and computer program products for implementing systems and methods for automated and scalable preprocessing of large numbers of documents for translation.

In various embodiments, a data processing system for automatically preprocessing of files for translation comprises a set of logic modules that are configured to access a set of original files to be translated, receive a set of attributes for the translation, generate automatically a set of pre-processed files based on at least one of the original files and based on at least one of the attributes, and make available at least one of the pre-processed files for translation.

In various embodiments, the automatic generation of the set of pre-processed files takes place without any direct action from any human.

In various embodiments, the attributes are included in an input supporting dataset.

In various embodiments, at least one of the following applies: the input supporting dataset includes metadata that corresponds to at least one of the attributes for the translation, and/or the input supporting dataset is included in, or is a metadata file.

In various embodiments, the input supporting dataset is included in, or is a companion file received directly or indirectly from a customer, and wherein the companion file defines at least one of the attributes for the translation.

In various embodiments, the companion file is received from a remote online system, via email, or through an application programming interface (API).

In various embodiments, the set of attributes for the translation includes at least one of the following: document type; ID for a template type; translation language combination; service type; source file name; and/or target file name.

In various embodiments, the service type includes at least one of the following: audio; braille; translation; transcreation; conversion to large print; conversation to standard print; or document enlargement.

In various embodiments, at least one of the translated files is translated using an AI engine.

In various embodiments, the AI was trained based on a training dataset that includes translated content curated by one or more humans.

In various embodiments, the AI engine is one of the following: a large language model (LLM); a neural network (NN) that is not an LLM; and/or a machine learning algorithm that is not an LLM or a NN.

In various embodiments, the number of preprocessed files exceeds one or more of the following during any period of twenty-four consecutive hours: 100; 1000; 2000; 4000; 6000; 8000; or 10000.

In various embodiments, the automatic generation of the set of pre-processed files includes at least one of the following: convert at least one of the original files from Portable Document Format (PDF) into a document format that is editable with non-PDF document processing software; store into a secure environment protected health information (PHI) that is included in at least one of the original files; decompress at least one of the original files; decrypt at least one of the original files; move to a different server at least one of the original files; transmit or receive through an application programming interface (API) at least one of the original files; validate at least one of the input files; create a new folder or subfolder, and place in the new folder or subfolder at least one of the original files, or at least one of the pre-processed files; compare at least one of the original files with at least one template, and identify at least one portion of the original file that is different compared to the template; compare at least one of the original files with content that was previously translated, and identify at least one portion of the original file that is different compared to the content that was previously translated; and/or store at least one original file in a storage memory.

In various embodiments, at least one pre-processed file includes protected health information (PHI), and wherein the at least one pre-processed file is made available for translation through an application programming interface (API) that is different from the API used to make available for translation pre-processed files that do not include PHI.

In various embodiments, a method for automatic translation is implemented on a data processing system comprising a set of logic modules that are configured to perform the following steps: access a set of original files to be translated; receive a set of attributes for the translation; generate automatically a set of pre-processed files based on at least one of the original files and based on at least one of the attributes; and make available at least one of the pre-processed files for translation.

This Summary section is provided to introduce a selection of concepts at a higher overview level, but is not specifically intended to identify key features, essential applications, or better implementations of the claimed subject matter. Consequently, nothing in this Summary section may be used to limit the scope of the claimed subject matter presented in the Claims.

### INCORPORATION BY REFERENCE

All publications, patents, and patent applications mentioned herein, if any, are incorporated by reference to the same extent as if each such individual publication, patent, or patent application were specifically and individually indicated to be incorporated by reference. To the extent that any inconsistency or conflict may exist between information expressly disclosed herein and information disclosed in any publications, patents, or patent applications that are incorporated by reference in this patent, the information expressly disclosed in this patent application (or patent, upon issuance) will take precedence and prevail.

### BRIEF DESCRIPTION OF THE DRAWINGS

The accompanying figures, which together with the detailed description below are incorporated in and form part of the specification, serve to further illustrate various embodiments and to explain various principles and advantages all in accordance with example embodiments of the present inventions.

FIG. 1 shows an exemplary architecture of a system for automatic processing of files 100 that can be used to preprocess one or more files in accordance with an embodiment.

FIG. 2 shows a representation of an exemplary data processing system 200 that may be used in connection with various embodiments and that may be configured to execute instructions for performing functions and methods described and/or claimed in connection with various embodiments.

FIG. 3 shows an exemplary flow diagram for a method or process for automatic translation 300 that can be used to translate one or more files in accordance with an embodiment.

FIG. 4 shows an exemplary flow diagram for a method or process for automatic processing 400 that can be used to automatically process one or more files in accordance with an embodiment.

FIG. 5 shows an exemplary flow diagram for a method or process for automatic processing 500 that can be used to automatically process one or more files in accordance with an embodiment.

### DETAILED DESCRIPTION

While the specification concludes with claims defining the features of the invention that are regarded as novel, the invention will be better understood from a consideration of the following description in conjunction with the drawing figures, in which like reference numerals are carried forward.

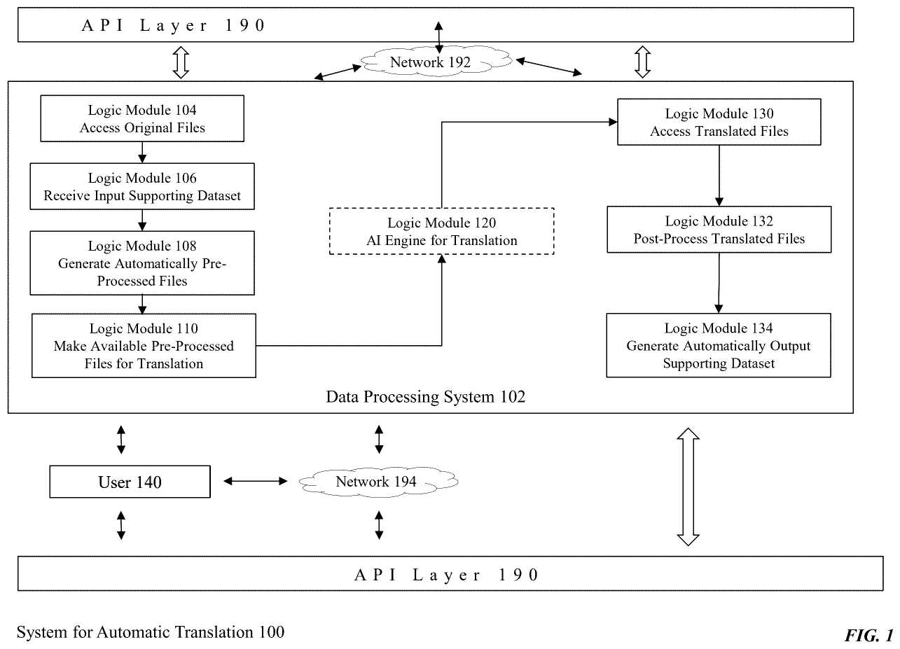

FIG. 1 shows an exemplary architecture of a system for automatic translation 100 in accordance with an embodiment.

The exemplary system for automatic translation 100 illustrated in the embodiment of FIG. 1 includes a data processing system 102 connected to a network 192, a network 194, and an API layer 190.

In various embodiments, the data processing system 102 includes several logic modules that are capable of working together to process, manage and translate large volumes of files automatically, without requiring direct human intervention at certain stages of the process.

A system that seeks to translate fast a large number of files should avoid reliance on human input in certain stages of the translation process because human input is likely to introduce delays compared to the processing speed that an automatic translation system could achieve while running on a typical high-power, high availability commercial cloud platform. Another risk with human input in a translation system is that humans could introduce errors into the translation process (e.g., in particular users with more limited training, or users who are under time pressure to complete high volumes of document translations), compared to the accuracy that an automated system can achieve if it has been properly programmed and calibrated to perform the translation process automatically.

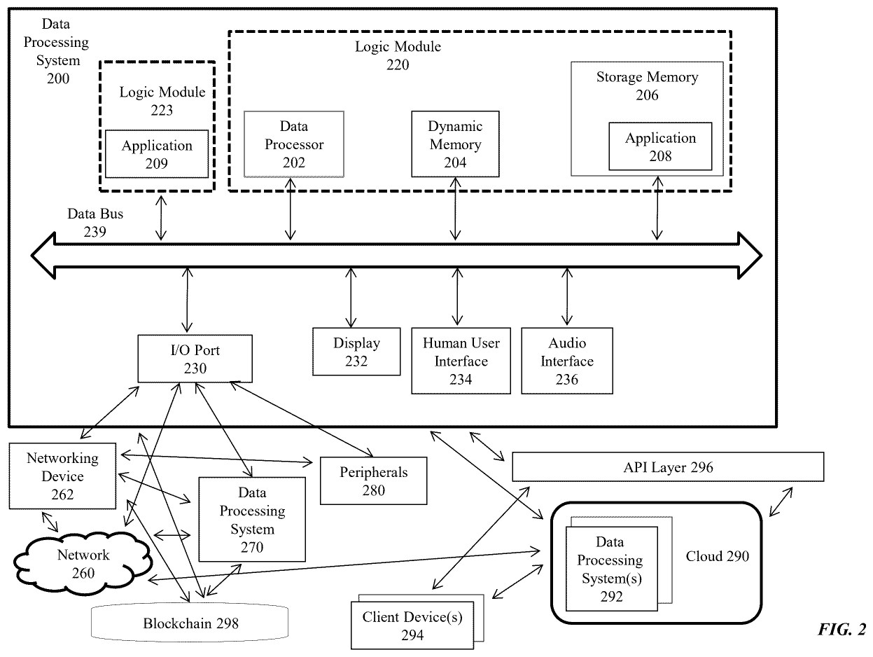

In the embodiment of FIG. 1, the data processing system 102 comprises logic modules 104, 106, 108, 110, 130, 132, and 134 that coordinate various stages of the translation process. In various embodiments, logic module 120 may also be included in the data processing system 102. In various embodiments, each of the logic modules 104, 106, 108, 110, 130, 132, and 134, and optional logic module 120, could be, or could include, logic module 220 and/or logic module 223 described in connection with the embodiments of FIG. 2.

In various embodiments, each of the logic modules 104, 106, 108, 110, 130, 132, and 134, and optional logic module 120, could include multiple logic modules, such as logic module 220 and/or logic module 223 described in connection with the embodiments of FIG. 2. For example, logic module 104 could be implemented as a combination of a hardware logic module and software logic module, as described in more detail in connection with logic module 220 and logic module 223.

In various embodiments, two or more of the logic modules 104, 106, 108, 110, 120 (which may be optionally included in data processing system 102), 130, 132, and 134, could be combined and could be implemented through fewer logic module. In one embodiment, a single logic module could include sufficient hardware and software to implement all of the logic modules 104, 106, 108, 110, 120 (which may be optionally included in data processing system 102), 130, 132, and 134.

In various embodiments, data processing system 102 is a cloud, or is deployed in a cloud. Clouds and cloud computing resources are discussed in more detail in connection with the embodiments of FIG. 2 below.

External networks 192 and 194 provide connectivity and facilitate data transmission between the data processing system 102 and user 140, and between the data processing system 102 and other external data processing systems, users or entities, such as translation service providers, customers, or cloud-based storage.

In various embodiments, logic module 104 may be configured to access a set of original files to be translated. In various embodiments, one or more of the original files could be provided by a customer, or could be received or retrieved from various storage systems, databases, or other repositories. In various embodiments, the logic module 104 may be configured to identify, retrieve, and/or make available or prepare the original files for pre-processing, ensuring that files are available in formats that can be handled during subsequent processing stages.

In various embodiments, the type of original files may include any suitable type, such as text documents, technical manuals, multimedia files, legal documents, medical documents, business documents, audio files, video files, images (e.g., images that include text and which could be processed to recognize the text through optical character recognition, multimodal AI engines, or other text recognition means), and any other types of documents or content, in any languages and/or formats supported by the data processing system 102. In various embodiments, the format of original files may include any suitable format, such as text (e.g., .txt or .doc) file, Portable Document Format (PDF), Microsoft Office (e.g., Word, Excel, PowerPoint, etc.) JPEG, JSON, XML, HTML, MPEG (e.g., any variation and successor of the MPEG audio and/or video standards), or any other type of text, image, video or audio file, or any other type of files that can be processed by data processing system 102. In various embodiments, the original files may be encrypted.

In various embodiments, logic module 106 is configured to receive an input supporting dataset. In various embodiments, the input supporting dataset may define various attributes for the translation. This input supporting dataset may include key information about the original files to be translated, such as language combinations (e.g., a source language and one or more languages in which the document must be translated), document type, service type, source file name, target file name (i.e., the name that the translated document will have after translation), template identification, decryption information (e.g., a decryption key), and other metadata that assists in tailoring the translation to specific needs.

In various embodiments, examples of service type include the following: conversion into or from an audio format, conversion between various audio formats, conversion into or from a video format, conversion between various video formats, conversion into or from a braille format, translation, transcreation (i.e., the process of adapting a message from one language to another while maintaining its intent, style, tone, and/or context), conversion into standard print, conversion into large print, any other document enlargement (e.g., into large print), and other such processes.

The input supporting dataset can be provided by external customers, defined within system configurations, or retrieved from prior stored datasets. In various embodiments, the input supporting dataset can be used to configure the translation system to handle the specific requirements of the translation project.

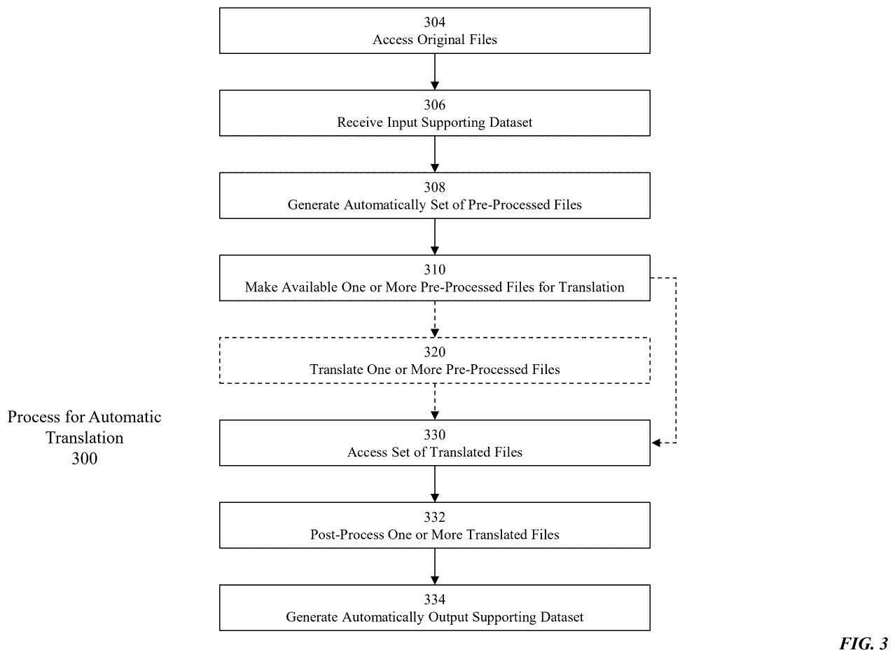

In various embodiments, the input supporting dataset may be included in, or may consist of an input metadata file. In various embodiments, the input supporting dataset may be included in, or may consist of a companion file. More information regarding an input supporting dataset and a companion file is provided in connection with Step 306 of FIG. 3.

In various embodiments, logic module 106 could receive the input supporting dataset through an application programming interface (API), such as API layer 190, or through a network (e.g., network 192 or 194).

In various embodiments, once the original files and input supporting dataset are retrieved, logic module 108 is configured to automatically generate a set of pre-processed files. In various embodiments, the pre-processed files are generated based on one or more of the original files. In various embodiments, the pre-processed files are generated based on the input supporting data. In various embodiments, the pre-processed files are generated based on both a set of the original files and based on the input supporting data.

In various embodiments, the automatic generation of the set of pre-processed files by logic module 108 can be implemented as discussed below in connection with step 308 of the embodiments of FIG. 3.

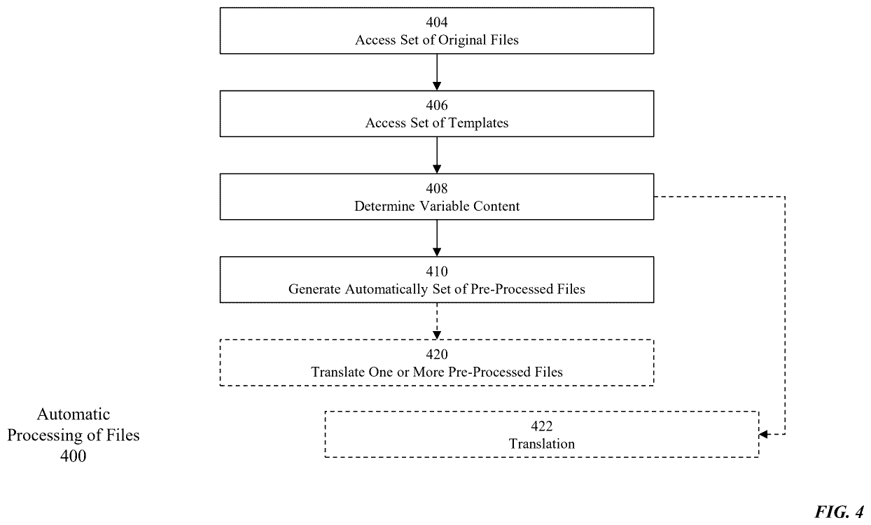

In various embodiments, the automatic generation of the set of pre-processed files by logic module 108 can be implemented as discussed below in connection with step 410 of the embodiments of FIG. 4.

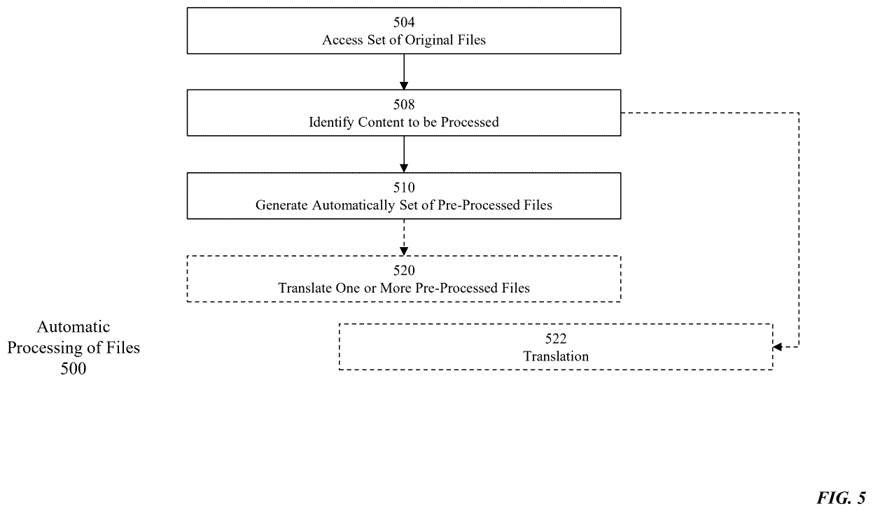

In various embodiments, the automatic generation of the set of pre-processed files by logic module 108 can be implemented as discussed below in connection with step 510 of the embodiments of FIG. 5.

In various embodiments, the input supporting dataset includes metadata that corresponds to at least one of the attributes for the translation. In various embodiments, the input supporting dataset is a metadata file.

In various embodiments, the input supporting dataset is a companion file received directly or indirectly from a customer (e.g., received from a customer, received through a vendor of a customer, etc.), and the companion file defines at least one attribute for the translation.

In various embodiments, the output supporting dataset includes metadata that corresponds to at least one of the translated files.

In various embodiments, the output supporting dataset is a metadata file.

In various embodiments, the automatic process to generate the set of pre-processed files may include one or more of the following operations:
(1) convert at least one of the original files from a format that cannot be easily edited (e.g., from Portable Document Format (PDF)) into a document format that is more easily editable (e.g., Microsoft® Word format, Google® Docs format, text format, etc.).
(2) store into a secure environment protected health information (PHI) that is included in at least one of the original files.
(3) decompress at least one of the original files.
(4) decrypt at least one of the original files.
(5) move to a different server at least one of the original files.
(6) transmit or receive through an application programming interface (API) at least one of the original files.
(7) validate at least one of the input files.
(8) create a new folder or subfolder, and place in the new folder or subfolder:
   (a) at least one of the original files, or
   (b) at least one of the pre-processed files;
(9) compare at least one of the original files with at least one template, and identify at least one portion of the original file that is different compared to the template;
(10) store at least one original file in a storage memory; and/or
(11) reproducing the original files or pre-processed files in the target language using one or more of the steps described above.

In various embodiments, the automatic generation of the set of pre-processed files takes place without any direct action from any human.

In various embodiments, one or more of the pre-processed files are subsequently made available by logic module 108 for translation by another logic module or by another data processing system. In various embodiments, one or more of the pre-processed files are made available by logic module 108 for translation by the logic module 120.

In various embodiments, one or more of the pre-processed files are subsequently made available by logic module 108 to logic module 110, and logic module 110 makes available one or more of such pre-processed files for translation by another logic module or by another data processing system. In various embodiments, one or more of the pre-processed files are made available by logic module 108 to logic module 110, and logic module 110 makes available one or more of such pre-processed files for translation by the logic module 120.

In various embodiments, one or more of the pre-processed files may contain sensitive information, such as Protected Health Information (PHI). In such cases, a pre-processed file may be handled through a specific API that offers enhanced security. In various embodiments, this API may be different from the API that is used to make available for translation pre-processed files that do not include PHI.

In various embodiments, the pre-processed files may be made available for translation through network-based APIs, allowing remote access by translation engines or other service providers, ensuring seamless integration into the workflow. In this embodiment, pre-processed files may be stored temporarily in secure environments before translation, and access is controlled through a set of predefined protocols.

In various embodiments, logic module 120 includes or uses an AI engine configured to translate one or more of the pre-processed files. In various embodiments, logic module 120 includes or uses two or more AI engines that are used together to translate one or more of the pre-processed files.

In various embodiments, each AI engine may be, or may include a large language model, (LLM), a neural network (NN), or any other machine learning algorithm capable of translating one or more of the pre-processed documents. AI engines are discussed in more detail in connection with the embodiments of FIG. 2 below.

In various embodiments, the logic module 120 is not part of the data processing system 102, and instead it is deployed separately. To illustrate this possibility, the border of the logic module 120 is shown as a dotted line in FIG. 1.

In various embodiments, logic module 120 may be deployed remotely from data processing system 102 (e.g., in a different cloud, in a server hosted on the premises of a business entity, or in a different data processing system). In various embodiments, the logic module 120 may communicate with the data processing system 102, with logic module 108, with logic module 110 and/or with logic module 130 through one or more APIs (such as API layer 190), one or more networks (such as network 192 and/or network 194), or through other communication channels.

In various embodiments, the logic module 120 is located remotely from data processing system 102, and the logic module 120 receives one or more preprocessed files that were generated by logic module 108 directly or indirectly, through one or more APIs (such as API layer 190), through one or more networks (such as network 192 and/or network 194), and/or through one or more other communication channels.

In various embodiments, the logic module 120 is located remotely from data processing system 102, and the logic module 120 translates one or more preprocessed files that were generated by logic module 108 to produce a set of translated files. In various embodiments, one or more translated files produced by the logic module 120 are made available to logic module 130 directly or indirectly, through one or more APIs (such as API layer 190), through one or more networks (such as network 192 and/or network 194), and/or through one or more other communication channels.

In various embodiments, the logic module 120 is included in the data processing system 102.

In various embodiments, the logic module 120 is included in the data processing system 102, receives a set of pre-processed files that were generated by logic module 108, and translates at least a subset of those pre-preprocessed files to produce a set of translated files. In various embodiments, one or more of the translated files are subsequently made available to the logic module 130.

In various embodiments, the logic module 120 includes or utilizes a large language model (LLM) to produce the translated documents. LLMs are discussed in more detail in connection with the embodiments of FIG. 2 below.

In various embodiments, the LLM was trained based on a training dataset that includes translated content curated by one or more humans (e.g., training datasets that may have been curated by human translators). Such curated content may help enhance the quality and accuracy of the translations performed by logic module 102, especially for technical, legal, medical, or other context-sensitive documents, and for certain languages.

When deployed commercially, a system that implements various aspects of the data processing system was able to translate thousands of documents per day. More generally, a translation system similar to the system for automatic translation 100 that is properly configured and runs with adequate computing resources could be expected to translate in excess of 100, 1000, 2000, 4000, 6000, 8000, or 10000 files during a 24-hour period, and any number of files in between the foregoing figures. Moreover, if the computing resources are high enough, the volume of files that can be processed by a translation system similar to the system for automatic translation 100 described in connection with the embodiments of FIG. 1 could increase indefinitely depending on the computing resources made available to such system.

In various embodiments, once the logic module 120 has translated at least a subset of the pre-processed files, it produces and makes available a set of translated documents to logic module 130 for further processing.

In various embodiments, logic module 130 accesses at least one of the translated files.

In various embodiments, logic module 132 post-processes at least one of the translated files. In various embodiments, the post-processing of the translated files may include at least one of the following:
(1) automatic quality assurance (QA) validation;
(2) moving or copying the file to a particular folder in response to an issue identified during the automatic QA validation process;
(3) changing the name of a file;
(4) review of at least one of the translated files by a human.
(5) modification of at least one of the translated files by a human;
(6) creating an automatic archive that includes at least one of the translated files;
(7) deleting at least one intermediate file that was created during the translation process (this may be helpful because the translation process could generate a significant number of intermediate files or other intermediate data, and these files or data should ideally be deleted to free up computing and memory resources);
(8) changing the name of a file (e.g., in response to a request made or rule set by a customer); and/or
(9) performing other steps to finalize a file before delivery to a client (e.g., packaging files, compressing files, encrypting files, and so on).

In various embodiments, logic module 134 generates automatically an output supporting dataset based on one or more of the translated files. In various embodiments, the output supporting dataset includes at least one of the following:
(1) invoice information related to the translation;
(2) a fee corresponding to at least one of the translated files; or
(3) reporting data for a customer based on at least one of the translated files.

In various embodiments, the automatic generation of the output supporting dataset takes place without any direct action from any human.

In various embodiments, the output supporting dataset may be transmitted to a customer or to another party by data processing system 102, or may be stored in a storage memory.

In various embodiments, network 192 and network 194 provide communication functionality for the data processing system 102. These networks may enable the transfer of files, datasets, and communications between the logic modules, data processing systems, and external entities. Network communication can occur through various protocols, including secure internet connections, private networks, or cloud-based networks, ensuring that data is transmitted efficiently and securely. These networks also support the communication between API layer 190 and external components, customers, vendors, and/or storage memory resources.

In various embodiments, each of the users 140 may communicate with data processing system 102 using a personal computer, laptop, mobile phone, mobile tablet, or any other data processing system, such as the data processing systems discussed in connection with FIG. 2. In various embodiments, one or more users 140 may have credentials that enable them to configure, monitor or otherwise use or interact with the data processing system 102.

In one embodiment, the API layer 190 from FIG. 1 may be, or may include the API layer 296 from the embodiment of FIG. 2. In various embodiments, the API layer 190 may include common and/or dedicated APIs that allow various components of the system for automatic translation 100 to receive data, including files to be translated. In various embodiments, the API layer 190 may facilitate data communications between various logic modules included in the data processing system 102, one or more users 140, and other communication endpoints.

In various embodiments, communications and file transfers may occur via an API layer, directly via a network without using an API (e.g., using network 192 and/or network 194 illustrated in the embodiment of FIG. 1), directly via a communication channel without using an API, and/or through any combination of an API, network and communication channel. For example, in various embodiments, communications through an API layer also traverses one or more networks (e.g., a WiFi network, a cellular network, etc.). Additional discussion of networks and communication channels is provided in connection with the embodiment of FIG. 2. As another example, in various embodiments, file transfers, or communications between the data processing system 102 and a data processing system in possession of a user 140 may take place through any of the following: (a) directly via one or more networks and without going through any APIs (e.g., a mobile app running on a consumer phone may establish an encrypted connection to an omnichannel layer and conduct a commerce transaction by transmitting and receiving data without going through an API), (b) through an API, and (c) through a combination of one or more APIs and directly via one or more networks and without going through an API (e.g., some communications may be direct and without an API, and some communications may be routed via an API).

FIG. 2 shows a representation of an exemplary data processing system 200 that may be used in connection with various embodiments and that may be configured to execute instructions for performing functions and methods described and/or claimed in connection with various embodiments. In various implementations, the data processing system 200 represents the data processing system 102 described in connection with the embodiments of FIG. 1 above, or any of the data processing systems used by users 140 to receive, transmit or process data in the embodiment of FIG. 1.

In various embodiments, the data processing system 200 may be an electronic tablet comprising a multi-touch display sensitive screen, a mobile phone, a wearable device, a vehicle entertainment system, a vehicle navigation system, a vehicle information system, or another mobile personal communication device. Examples of electronic tablets in accordance with various embodiments include an iPad tablet computer currently commercialized by Apple Inc. and running an iOS operating system, a tablet computer running the Android operating system currently developed by Google Inc, a tablet running the Windows operating system, and any other electronic tablet devices. Examples of mobile phones in accordance with various embodiments include a mobile phone using an iOS operating system (iPhone), a mobile phone using an Android operating system, a mobile phone using a Windows operating system, and other mobile phones. Examples of wearable devices in accordance with various embodiments include a watch with an electronic display, and an electronic eyewear device (e.g., electronic glasses such as Google Glass or other devices with a similar form factor). In various embodiments, an electronic tablet or a mobile phone is adapted to run one or more mobile apps that perform various functions. Examples of a vehicle entertainment system, vehicle navigation system, or vehicle information system in accordance with various embodiments includes any device that can relay visual or auditory information to a driver or passenger in a vehicle or other transportation device (e.g., car, bus, train, plane, ship, subway, elevator, etc.), including for example a car entertainment system that can display or recite to a driver or a passenger information about a shopping menu, product or service.

The exemplary data processing system 200 includes a data processor 202. The data processor 202 represents one or more general-purpose data processing devices such as a microprocessor or other central processing unit. More particularly, the processing device may be a complex instruction set computing (CISC) microprocessor, a reduced instruction set computing (RISC) microprocessor, a very long instruction word (VLIW) microprocessor, a processor implementing other instruction sets, or a processor implementing a combination of instruction sets, whether in a single core or in a multiple core architecture. Data processor 202 may also be or include one or more special-purpose processing devices such as an application specific integrated circuit (ASIC), a field programmable gate array (FPGA), a digital signal processor (DSP), network processor, any other embedded processor, or the like. The data processor 202 may execute instructions for performing operations and steps in connection with various embodiments of the present invention. In various implementations, data processor 202 may be based on an ARM architecture commercialized by ARM Limited, x86, x32, x64 or subsequent architectures commercialized by Intel Corporation, x86-64 or subsequent architectures commercialized by Advanced Micro Devices, Inc., and/or on other processor architectures suitable that provide desirable attributes of performance, size, power consumption, packaging, features, cost, and/or other characteristics. In some embodiments (e.g. for mobile device applications), data processor 202 may be, may be included in, or may include a system on a chip (SoC) design comprising one or more CPU cores, one or more graphics processing unit (GPU), one or more wireline or wireless modems, one or more global positioning system (GPS) modules, camera functionality, gesture recognition functionality, video functionality, and/or other software and hardware features.

In some embodiments, data processor 202 may be configured to, adapted to, or optimized for performing artificial intelligence (AI) functionality, such as processing any large language model (LLM), neural network models, or other machine learning algorithms.

In some embodiments, the data processor 202 may include specialized hardware for AI tasks, such as tensor processing units (TPUs), graphics processing units (GPUs), neural processing units (NPUs), or other AI accelerators designed for high-performance matrix operations, which may facilitate training and inference tasks in deep learning models. In some embodiments, data processor 202 may support parallelized operations across multiple cores or hardware threads, vectorization of computations via instruction sets such as AVX (Advanced Vector Extensions), AVX-512, or other SIMD (Single Instruction, Multiple Data) capabilities, and/or may be capable of performing complex operations such as backpropagation, stochastic gradient descent (SGD), and other functions or algorithms for training machine learning models. In some embodiments, data processor 202 may support AI-specific frameworks such as TensorFlow, PyTorch, or other machine learning platforms, to support deployment and scaling of AI models. In some cases, the processor may include dedicated memory hierarchies or caches optimized for high-bandwidth data access, reducing latency during AI-related tasks, and may also feature energy-efficient design to handle intensive workloads while minimizing power consumption in edge devices or mobile applications.

In the exemplary embodiment of FIG. 2, the data processing system 200 further includes a dynamic memory 204, which may be designed to provide higher data read speeds. Examples of dynamic memory 204 include dynamic random access memory (DRAM), synchronous DRAM (SDRAM) memory, read-only memory (ROM) and flash memory. The dynamic memory 204 may be adapted to store all or part of the instructions of a software application, as these instructions are being executed or may be scheduled for execution by data processor 202. In some implementations, the dynamic memory 204 may include one or more cache memory systems that are designed to facilitate lower latency data access by the data processor 202.

In this exemplary embodiment, the data processing system 200 further includes a storage memory 206, which may be designed to store larger amounts of data. Examples of storage memory 206 include a magnetic hard disk and a flash memory module. In various implementations, the data processing system 200 may also include, or may otherwise be configured to access one or more external storage memories, such as an external memory database or other memory data bank, which may either be accessible via a local connection (e.g., a wired or wireless USB, Bluetooth, or WiFi interface), or via a network (e.g., a remote cloud-based memory volume).

A storage memory may also be denoted a memory medium, storage medium, dynamic memory, or memory. In general, a storage memory, such as the dynamic memory 204 and the storage memory 206, may include any chip, device, combination of chips and/or devices, or other structure capable of storing electronic information, whether temporarily, permanently or quasi-permanently. A memory medium could be based on any magnetic, optical, electrical, mechanical, electromechanical, MEMS, quantum, or chemical technology, or any other technology or combination of the foregoing that is capable of storing electronic information. A memory medium could be centralized, distributed, local, remote, portable, or any combination of the foregoing. Examples of memory media include a magnetic hard disk, a random access memory (RAM) module, an optical disk (e.g., DVD, CD), and a flash memory card, stick, disk or module.

A software application or module, and any other computer executable instructions, may be stored on any such storage memory, whether permanently or temporarily, including on any type of disk (e.g., a floppy disk, optical disk, CD-ROM, and other magnetic-optical disks), read-only memory (ROM), random access memory (RAM), EPROM, EEPROM, magnetic or optical card, or any other type of media suitable for storing electronic instructions.

In general, a storage memory could host a database, or a part of a database. Conversely, in general, a database could be stored completely on a particular storage memory, could be distributed across a plurality of storage memories, or could be stored on one particular storage memory and backed up or otherwise replicated over a set of other storage memories. Examples of databases include operational databases, analytical databases, data warehouses, distributed databases, end-user databases, external databases, hypermedia databases, navigational databases, in-memory databases, document-oriented databases, real-time databases and relational databases.

Storage memory 206 may include one or more software applications 208, in whole or in part, stored thereon. In general, a software application, also denoted a data processing application or an application, may include any software application, software module, function, procedure, method, class, process, or any other set of software instructions, whether implemented in programming code, firmware, or any combination of the foregoing. A software application may be in source code, assembly code, object code, or any other format. In various implementations, an application may run on more than one data processing system (e.g., using a distributed data processing model or operating in a computing cloud), or may run on a particular data processing system or logic module and may output data through one or more other data processing systems or logic modules.

The exemplary data processing system 200 may include one or more logic modules 220 and/or 221, also denoted data processing modules, or modules. Each logic module 220 and/or 221 may consist of (a) any software application, (b) any portion of any software application, where such portion can process data, (c) any data processing system, (d) any component or portion of any data processing system, where such component or portion can process data, and (e) any combination of the foregoing. In general, a logic module may be configured to perform instructions and to carry out the functionality of one or more embodiments of the present invention, whether alone or in combination with other data processing modules or with other devices or applications. Logic modules 220 and 221 are shown with dotted lines in FIG. 2 to further emphasize that data processing system 200 may include one or more logic modules, but does not have to necessarily include more than one logic module.

As an example of a logic module comprising software, logic module 223 shown in FIG. 2 consists of application 209, which may consist of one or more software programs and/or software modules. Logic module 223 may perform one or more functions if loaded on a data processing system and/or on a logic module that comprises a data processor.

As an example of a logic module comprising software, in the embodiment of FIG. 2, logic module 223 includes application 209. In various embodiments, application 209 may be, or may include a set of software programs and/or a set of software modules. Logic module 223 and/or application 209 may include various functionality, and/or may perform various functions when running on a data processing system and/or on a logic module that comprises a data processor.

Examples of functionality that may be included in, and/or may be provided by logic module 223 and/or application 209 include, in various embodiments, the following:
(1) Operating system functionality, such as managing hardware resources, file systems, user interfaces, and application execution. Examples of such operating systems may include Windows, macOS, Linux, Android, iOS, and cloud-based operating systems such as Google's Chrome OS or Amazon's Fire OS for cloud computing environments;
(2) Client computer (e.g., desktop, laptop, etc.) software functionality, such as document editing, database management, and internet browsing. Examples of such software functionality may include word processing (e.g., Microsoft Word), spreadsheet applications (e.g., Microsoft Excel), database management systems (e.g., MySQL, Oracle), and internet browsers (e.g., Google Chrome, Firefox);
(3) Mobile device (e.g., mobile phone, wearable device, etc.) software functionality, such as messaging, social media interaction, and navigation. Examples of such software functionality may include word processing (e.g., Google Docs), navigation apps (e.g., Google Maps, Waze), fitness tracking apps (e.g., Fitbit, Apple Health), and mobile payment systems (e.g., Google Pay, Apple Pay);
(4) Cloud software functionality, such as Software as a Service (SaaS), Platform as a Service (PaaS), and Infrastructure as a Service (IaaS). Examples of such software functionality may include email services (e.g., Gmail), e-commerce platforms (e.g., Shopify, Amazon Web Services), translation or interpretation services for documents and/or other content (e.g., audio, video, images, or other types of content), and virtualized development environments (e.g., Microsoft Azure, Heroku);
(5) Artificial intelligence (AI) functionality. Examples of such AI functionality may include natural language processing (NLP), vectorization of data, inference engines, training of machine learning models, image recognition, speech-to-text conversion, and recommendation algorithms.

In various embodiments, AI functionality may include and/or may be based on data preprocessing tasks such as data normalization, feature extraction, and noise reduction, which prepare raw data for more accurate and efficient machine learning or natural language processing (NLP) operations. These preprocessing tasks may involve automated data cleansing, deduplication, and tagging, enabling the system to organize and format large datasets for downstream AI processing. In the context of document management, AI functionality may include automated document classification, metadata extraction, content summarization, and indexing, facilitating efficient retrieval, searchability, and categorization of documents.

In various embodiments, an “AI engine” can be broadly described as a computational framework or system designed to provide artificial intelligence functionality by automating tasks that typically require human cognition, such as decision-making, learning, language processing, and pattern recognition. In various embodiment, AI engines can be implemented using a variety of technologies, which may offer varying capabilities for specific types of tasks.

In some embodiments, an AI engine may be implemented through one or more large language models (LLMs). In various embodiments, LLMs can be used for natural language processing (NLP) tasks. LLMs, such as OpenAI's GPT series or Google's BERT, are based on transformer architectures that enable contextual understanding of language, making them helpful for tasks like text generation, translation, and summarization.

In some embodiments, an AI engine may be implemented through neural networks (NNs), which are a broader class of machine learning models inspired by the structure of biological neurons. In some implementations, LLMs may be considered a subclass of NNs. In various embodiments, neural networks may be configured in various ways, such as convolutional neural networks (CNNs) for image recognition or recurrent neural networks (RNNs) for sequence data.

In various embodiments, LLMs and other NNs may be useful for complex, high-dimensional data tasks, but can be computationally intensive.

In some embodiments, an AI engine may be implemented through one or more machine learning algorithms, such as decision trees, support vector machines (SVMs), or clustering techniques, which may offer simpler and less computationally-intensive alternatives to LLMs. These machine learning algorithms may not require deep learning architectures, and may be used for tasks like classification, regression, and clustering on structured data.

In various embodiments, an AI engine can be implemented using a variety of AI technologies, with each approach offering distinct advantages based on the complexity of the task, the nature of the data, and the desired balance between interpretability and computational efficiency.

In various embodiments, one or more AI engines may provide AI functionality for various data and content management tasks. For example, in one implementation, an AI engine could be an LLM, and the LLM may be employed for real-time translation of documents across multiple languages, ensuring accurate and contextually appropriate translations using AI-driven algorithms. In various embodiments, writing augmentation functionality may leverage LLMs to assist with content generation, document drafting or editing, and content suggestions, where the AI engine can generate text, recommend edits, or enhance writing style based on contextual analysis of the input text.

In various embodiments, an AI engine may provide advanced functionality such as entity recognition, sentiment analysis, and topic modeling, which enhance document understanding and facilitate more effective content management. These capabilities can be integrated into workflow systems for legal, medical, or technical document generation, translation, review, or management, which may help provide consistency, accuracy, and compliance with domain-specific standards. In various embodiments, an AI engine may also include adaptive learning algorithms that improve over time through feedback and usage patterns, further refining document management tasks, such as automatic organization, version control, and collaboration support.

In various embodiments, an AI engine may include and/or may leverage a wide range of large language models (LLMs) and/or other AI technologies. Examples of LLMs include OpenAI's GPT series (e.g., GPT-4), Google's BERT (Bidirectional Encoder Representations from Transformers), T5 (Text-to-Text Transfer Transformer), and PaLM (Pathways Language Model). These models are able to process, generate, and understand human language to an extent, and they may be used for tasks such as text generation, translation, summarization, and content analysis. Additionally, AI technologies like reinforcement learning, convolutional neural networks (CNNs), and transformers are used for image recognition, video analysis, and pattern detection. In some cases, hybrid AI systems may combine LLMs with specialized AI engines, such as those designed for optical character recognition (OCR), voice recognition (e.g., Google's WaveNet), or computer vision (e.g., OpenAI's CLIP). These models and technologies can be deployed to handle a wide array of document and content management tasks, providing scalability and versatility in AI-driven applications.

In various embodiments, one or more AI engines can provide AI functionality to perform translation and/or interpretation across multiple languages. AI functionality provided by AI engines for translation and/or interpretation may leverage LLMs and/or may use deep learning techniques and contextual understanding to provide improved translations compared to non-AI systems (e.g., more accurate, more nuanced, etc.). In various embodiments, an AI engine may analyze longer text blocks (e.g., entire sentences or paragraphs) to understand the context, enabling the production of more coherent and more grammatically correct translations. Such AI systems can be employed for real-time or expedited translation in multilingual document management environments, therefore enabling faster conversion of text from one language to another while better preserving meaning, tone, and/or style. In various embodiments, an AI engine can be trained based on datasets of bilingual text or other bilingual content (e.g., audio, video, or images), enabling it to better process idiomatic expressions, cultural references, and domain-specific jargon (e.g., legal, financial, or medical terms), and therefore increasing the accuracy and contextual appropriateness of translations and/or interpretations provided by that AI engine.

As an example of a logic module comprising hardware, the logic module 220 shown in FIG. 2 comprises data processor 202, dynamic memory 204 and storage memory 206. Examples of data processing systems that may incorporate both logic modules comprising software and logic modules comprising hardware include a desktop computer, a mobile computer, a server computer, or a cloud, each being capable of running software to perform one or more functions defined in the respective software.

In general, functionality of logic modules may be consolidated in fewer logic modules (e.g., in a single logic module), or may be distributed among a larger set of logic modules. For example, separate logic modules performing a specific set of functions may be equivalent with fewer or a single logic module performing the same set of functions. Conversely, a single logic module performing a set of functions may be equivalent with a plurality of logic modules that together perform the same set of functions. In the data processing system 200 shown in FIG. 2, logic module 220 and logic module 223 may be independent modules and may perform specific functions independent of each other. In an alternative embodiment, logic module 220 and logic module 223 may be combined in whole or in part in a single module that perform their combined functionality. In an alternative embodiment, the functionality of logic module 220 and logic module 223 may be distributed among any number of logic modules. One way to distribute functionality of one or more original logic modules among different substitute logic modules is to reconfigure the software and/or hardware components of the original logic modules. Another way to distribute functionality of one or more original logic modules among different substitute logic modules is to reconfigure software executing on the original logic modules so that it executes in a different configuration on the substitute logic modules while still achieving substantially the same functionality. Examples of logic modules that incorporate the functionality of multiple logic modules and therefore can be construed themselves as logic modules include system-on-a-chip (SoC) devices and a package on package (PoP) devices, where the integration of logic modules may be achieved in a planar direction (e.g., a processor and a storage memory disposed in the same general layer of a packaged device) and/or in a vertical direction (e.g. using two or more stacked layers).

The exemplary data processing system 200 may further include one or more input/output (I/O) ports, illustrated in FIG. 2 as I/O port 230, for communicating with other data processing system (e.g., data processing system 270), with other peripherals (e.g., peripherals 280), or with one or more networks (e.g., network 260). Each I/O port 230 may be configured to operate using one or more wired and/or wireless communication protocols, such as, for example, any protocols available in network 260, any protocols available to connect directly or indirectly to another data processing system such as data processing system 270, and/or any protocols available to connect directly or indirectly to peripherals such as Peripherals 280. In general, each I/O port 230 may be able to communicate through one or more communication channels and/or to connect to one or more networks, such as network 260 as illustrated in FIG. 2. The data processing system 200 may communicate directly with other data processing systems, such as data processing system 270 (e.g., via a direct wireless or wired connection), or via the one or more networks 260.

A communication channel or data network may include any direct or indirect data connection path, including any connection using a wireless technology (e.g., Bluetooth, infrared, WiFi, WiMAX, cellular, 3G, 4G, EDGE, CDMA and DECT), any connection using wired (also sometimes denoted “wireline”) technology (including via any serial, parallel, wired packet-based communication protocol (e.g., Ethernet, USB, FireWire, etc.), or other wireline connection), any optical channel (e.g., via a fiber optic connection or via a line-of-sight laser or LED connection), and any other point-to-point connection capable of transmitting data.

Each of the networks 260 may include one or more communication channels. In general, a network, or data network, consists of one or more communication channels that can be established between devices connected to each other directly or indirectly through that network. Examples of networks include a LAN, MAN, WAN, cellular and mobile telephony network, the Internet, the World Wide Web, and any other information transmission network. In various implementations, the data processing system 200 may include additional interfaces and communication ports in addition to the I/O Port 230.

In various embodiments, a network, such as network 260, may include a collection of terminal nodes, links and any intermediate nodes. A network maybe wired or wireless. An example of a wired network is an Ethernet network. An example of a wireless network is a WiFi network.

An example of a short-distance communication channel or network are near-field communication (NFC) applications, which are employed in some mobile devices to automate device-to-device transactions, such as payments, data synchronization, and other information exchange. Another example of a short-distance communication channel or network are radio frequency identification (RFID) data transfers that can be used to identify individual items using low-power communications (e.g., merchandize identification, automatic inventory, etc.).

In one embodiment, the data processing system 200 comprises a wireless communication module that enables the data processing system 200 to communicate wirelessly via network 260, using a wireless data protocol made available in the network 260 (e.g., a WiFi protocol). The network 260 may include both wireless and wireline connections (e.g., may permit communications using both WiFi and Ethernet protocols). In one embodiment, the network 260 may consist of two or more networks, whether wireless or wired, and the two or more networks may operate independently (e.g., to increase security by separating communications) or may be connected to each other (e.g., to facilitate communications among devices connected to different networks).

In one embodiment, the data processing system 200 is located in a particular facility (e.g., in a commercial establishment), and the network 260 represents a combination of an internal network deployed within that facility and an external communication channel or network that provides a connection to the Internet. In one embodiment, the data processing system 200 could be connected directly to the Internet through the network 260, could be connected to the Internet through an intermediate data processing system that acts as a gateway, or could be connected to the Internet through one or more networking devices, such as networking device 262 illustrated in FIG. 2 (e.g., a router, a modem, a gateway). In one embodiment, the data processing system 200 could be connected directly or indirectly to the Internet through the network 260 and could act as a gateway for one or more other data processing systems (e.g., other computing devices, peripheral devices used in point of sale or other commerce transactions, etc.).

In one embodiment, the data processing system 200 may communicate with a cloud or other remote data processing system via the network 260. In various embodiments, the cloud or other remote data processing system may assist the data processing system 200 to conduct or facilitate a commercial transaction (e.g., authenticating a user or a payment method, conducting or mediating a payment transaction, collecting or returning data or analytical information about a consumer, etc.).

In various embodiments, the network 260 is, or includes a network that facilitates communications at longer distances. In various embodiments, the network 260 is, or includes, a 3G network, a 4G network, an EDGE network, a CDMA network, a GSM network, a 3GSM network, a GPRS network, an EV-DO network, a TDMA network, an iDEN network, a DECT network, a UMTS network, a WiMAX network, a cellular network, any type of wireless network that uses a TCP/IP protocol or other type of data packet or routing protocol, any other type of wireless wide area network (WAN) or wireless metropolitan area network (MAN), or a satellite communication channel or network. Each of the foregoing types of networks that could be used within the network 260 utilizes various communication protocols, including protocols for establishing connections, transmitting and receiving data, handling various types of data communications (e.g., voice, data files, HTTP data, images, binary data, encrypted data, etc.), and otherwise managing data communications. In various embodiments, the data processing system 200 is configured to be compatible with one or more protocols used in the network 260, such that the data processing system 200 can successfully connect to the network 260 and communicate via the network 260.

The exemplary data processing system 200 may further include a display 232, which provides the ability for a user to visualize data output by the data processing system 200 and/or to interact with the data processing system 200. The display 232 may directly or indirectly provide a graphical user interface (GUI) adapted to facilitate presentation of data to a user and/or to accept input from a user. The display 232 may consist of a set of visual displays (e.g., an integrated LCD, LED or CRT display), a set of external visual displays, (e.g., an LCD display, an optical projection device, a holographic display), or of a combination of the foregoing.

A visual display may also be denoted a graphic display, computer display, display, computer screen, screen, computer panel, or panel. Examples of displays include a computer monitor, an integrated computer display, electronic paper, a flexible display, a touch panel, a transparent display, and a three dimensional (3D) display that may or may not require a user to wear assistive 3D glasses.

A data processing system may incorporate a graphic display. Examples of such data processing systems include a laptop, a computer pad or notepad, an electronic tablet or other tablet computer, a smart phone or any other mobile phone, an electronic reader (also denoted an e-reader or ereader), a personal data assistant (PDA), a medical device, or any other device that incorporates data processing features and a display for displaying information and/or receiving information from a user.

A data processing system may be connected to an external graphic display. Examples of such data processing systems include a desktop computer, a server, an embedded data processing system, a mobile phone, an electronic tablet, or any other data processing system adapted to display information through an external display, whether or not it includes a display itself. A data processing system that incorporates a graphic display may also be connected to an external display. A data processing system may directly display data on an external display, or may transmit data to other data processing systems or logic modules that will eventually display data on an external display.

Graphic displays may include active display, passive displays, LCD displays, LED displays, OLED displays, plasma displays, and any other type of visual display that is capable of displaying electronic information to a user. Such graphic displays may permit direct interaction with a user, either through direct touch by the user (e.g. a touch-screen display that can sense a user's finger touching a particular area of the display), through proximity interaction with a user (e.g., sensing a user's finger being in proximity to a particular area of the display), or through a stylus or other input device. In one implementation, the display 232 is a touch-screen display that displays a human GUI interface to a user, with the user being able to control the data processing system 200 through the human GUI interface, or to otherwise interact with, or input data into the data processing system 200 through the human GUI interface. Examples of touch-screen display technologies include resistive, surface acoustic wave, capacitive, infrared, optical imaging, dispersive signal, and acoustic pulse recognition

The exemplary data processing system 200 may further include one or more human input interfaces 214, which facilitate data entry by a user or other interaction by a user with the data processing system 200. Examples of human input devices 214 include a keyboard, a mouse (whether wired or wireless), a stylus, other wired or wireless pointer devices (e.g., a remote control), or any other user device capable of interfacing with the data processing system 200. In some implementations, human input devices 214 may include one or more sensors that provide the ability for a user to interface with the data processing system 200 via voice, or provide user intention recognition technology (including optical, facial, or gesture recognition), or gesture recognition (e.g., recognizing a set of gestures based on movement via motion sensors such as gyroscopes, accelerometers, magnetic sensors, optical sensors, etc.).

The exemplary data processing system 200 may further include one or more gyroscopes, accelerometers, magnetic sensors, optical sensors, or other sensors that are capable of detecting physical movement of the data processing system. Such movement may include larger amplitude movements (e.g., a device being lifted by a user off a table and carried away or elevation changes experienced by the data processing system), smaller amplitude movements (e.g., a device being brought closer to the face of a user or otherwise being moved in front of a user while the user is viewing content on the display, movement experienced by a vehicle within which the data processing system is located), or higher frequency movements (e.g., hand tremor of a human, vibrations caused by an engine). In the absence of internal motion sensors, or in addition to any internal motion sensors, the exemplary data processing system 200 may further be capable of receiving and processing information from external motion sensors such as gyroscopes, accelerometers, magnetic sensors, optical sensors, or other sensors that are capable of detecting physical movement of the data processing system.

The exemplary data processing system 200 may further include an audio interface 216, which provides the ability for the data processing system 200 to output sound (e.g., a speaker), to input sound (e.g., a microphone), or any combination of the foregoing.

The exemplary data processing system 200 may further include any other components that may be advantageously used in connection with receiving, processing and/or transmitting information.

In the exemplary data processing system 200, the data processor 202, dynamic memory 204, storage memory 206, I/O port 230, display 232, human input interface 214, audio interface 216, and logic module 221 communicate to each other via the data bus 219. In some implementations, there may be one or more data buses in addition to the data bus 219 that connect some or all of the components of data processing system 200, including possibly dedicated data buses that connect only a subset of such components. Each such data bus may implement open industry protocols (e.g., a PCI or PCI-Express data bus), or may implement proprietary protocols.

In one embodiment, a data processing system (such as data processing system 200) is connected to a networking device, illustrated in FIG. 2 as networking device 262. In various embodiments, the networking device 262 could act as a router (wireless and/or wired), hub, switch, modem, bridge, repeater, gateway, communication protocol converter, communication buffering device, or virtually any other type of equipment that can perform a networking or communication function. In various embodiments, the networking device 262 could perform various functions for data processing system 200, including acting as a connecting hub to other data processing systems (e.g., data processing system 270) and/or peripherals (e.g., one or more peripherals 280), providing a layer of security (e.g., acting as a firewall, providing a connection or user authentication layer, etc.), extending the range of a wireless communication channel or network (e.g., in a restaurant or other commercial establishment where data processing systems and peripherals are far from each other or are separated by metallic objects or thick walls), establishing a short-distance network (e.g., a BlueTooth network or other network intended to operate using low power or to provide physical security by limiting the effective connection range), and so on. In various embodiments, the networking device 262 may be adapted to communicate using a wired connection, such as a serial connection, a wired packet-based communication protocol (e.g., Ethernet, USB, FireWire, etc.), a parallel connection, and/or any other wireline protocol. In various embodiments, the networking device 262 may be adapted to communicate using a wireless connection, such as a WiFi connection or cellular network connection.

In one embodiment, the networking device 262 is adapted to handle data communications via a local network (e.g., network 260 in FIG. 2 could represent a local network, such as a WiFi network) with one or more local data processing systems and/or peripheral devices, such as the data processing system 270 and the peripheral devices 280. In one embodiment, the networking device 262 establishes a local network, and one or more of the data processing system 200, data processing system 270, and/or peripheral devices 280 join this local network. In another embodiment, a local network (e.g., network 260) is established by another device (e.g., by another wireless and/or wired router, by a data processing system, by a peripheral device, etc.), and the networking device 262, and one or more of the data processing system 200, data processing system 270, and/or peripheral devices 280 join this local network.

In various embodiments, a local network is a wireless network that facilitates wireless communications between devices that are deployed in a local configuration, for example being collocated within a room, building, facility or location. For example, a local network (e.g., network 260 in FIG. 2 could represent a local network) may be created within a restaurant, store, other retail location, hotel, gas station, school, employment location, or other business facility or commercial establishment, where a sale transaction or other commercial transaction could take place using one or more of the data processing system 200, data processing system 270, and/or peripheral devices 280.

In various embodiments, network 260 in FIG. 2 could represent a set of local networks, which could be, or could include, wireless and/or wired communication. In one embodiment, a local network could be a WiFi network, including any wireless network compliant with an IEEE 802.11 standard, any wireless network for local wireless communications developed by or with the assistance of the WiFi Alliance or other standard bodies or industry groups, or any other wireless local area network. In general, for various embodiments, it is desirable for a local network to be capable of establishing reliable wireless connections between two or more data processing devices and/or peripherals, even if they are not in immediate proximity.

In various embodiments, one or more data processing systems, such as the data processing system 200 of FIG. 2, may be connected directly or indirectly to a computational cloud, such as cloud 290 illustrated in FIG. 2. A computational cloud, also denoted a “cloud,” is a set of computing servers that provide computational capability, data storage and/or services capability to one or more client devices. The client devices are typically remote from the cloud and are accessing the cloud via one or more data networks. A cloud may include sophisticated computing and data storage capabilities, including advanced security, performance management, high reliability, redundancy, interoperability with various types of client devices (e.g., various types of data processing systems using different hardware and software configurations could connect to the same cloud and receive similar services), quick and cost-effective computing power provisioning, and so on. For example, in various embodiments, one or more of the data processing system 200 and the data processing system 270 may be different types of electronic tablets, mobile phones, laptops or personal computers, and they could both be connected to the cloud 290 via one or more networks (e.g., network 260 and/or other data networks). In one embodiment, one or more of the peripheral devices 280 may also be connected to the cloud 290.

A cloud, such as cloud 290, may provide access to various types of services. Services and functionality made available by clouds include Software as a Service (SAAS), Platform as a Service (PAAS), cloud computing, Infrastructure as a Service (IAAS), cloud storage, Internet-based computing, and so on. Depending on their characteristics, clouds may be classified as private clouds, public clouds, hybrid clouds, and so on.

In various embodiments, the data processing system 200 of FIG. 2 may be deployed as part of a cloud (i.e., may perform data processing functionality within the cloud). In various embodiments, the data processing system 200 may operate alone or together with other identical, similar or different data processing systems to provide cloud functionality, such as the cloud functionality described above in connection with cloud 290. In some embodiments, a cloud may include a large number of data processing systems (such as data processing system 200), data processors (such as data processor 202), and/or logic modules (such as logic module 220 or logic module 223). Examples of clouds that include a large number of data processing systems, data processors, and/or logic modules are data farms, distributed computing networks, or hyperscale data centers, which may be managed by cloud service providers such as Amazon Web Services (AWS), Microsoft Azure, Google Cloud Platform (GCP), or other public, private, or hybrid cloud infrastructures. These clouds may support large-scale computing tasks, including big data analytics, artificial intelligence (AI) workloads, content delivery, and various forms of software as a service (SaaS), platform as a service (PaaS), or infrastructure as a service (IaaS).

In various embodiments, one or more Application Processing Interfaces (APIs), such as the API layer 296 illustrated in FIG. 2, may be deployed to facilitate communications between data processing systems, clouds, networks, and/or other systems or components. In general, an API may be or may include a set of subroutine definitions, protocols, and/or tools for building or interconnecting web-based systems, operating systems, database systems, hardware and/or software. API specifications may include specifications for object classes, routines, variables, data structures, and/or remote calls. An advantage of using APIs to interconnect web-based systems, operating systems, database systems, hardware and/or software may include the ability to provide a common communication framework capable of communicating with different types of hardware software or technologies (e.g., data processing systems with Android, iOS, Windows and other Operating Systems may communicate with each other, web systems running on different software environments may exchange data using common protocols, etc.). Another advantage of using APIs to interconnect web-based systems, operating systems, database systems, hardware and/or software may include introducing additional layers of security for the components connected to the API (e.g., by alleviating the need to log in directly into a remote system, by limiting the features or types of data that can be exchanged, by limiting the ability to push v. pull data from a web system or server, etc.). Another advantage of using APIs to interconnect web-based systems, operating systems, database systems, hardware and/or software may include improved scalability of communications (e.g., as the volume of calls through an API increases, additional computing resources can be provisioned on demand to process such calls, etc.).

As shown in the embodiment of FIG. 2, the API layer 296 can be used to interface a variety of different components and systems, such as the data processing system 200, the data processing system 270, the peripherals 280, one or more client device 294, and/or the cloud 290. In general, a wide range of data processing systems or components with communication capabilities could be connected to an API layer using an appropriate protocol and/or data conversion.

Web APIs are a particular class of APIs that provide functionality for interfacing data processing systems, clouds and other servers capable of communicating via the Web or Internet. A Web API may provide an interface through which interactions happen between an enterprise and applications that use its assets. When deployed as a Web API, an API such as the API layer 296 may provide a programmable interface between a set of services and a set of applications serving different types of consumers. When used in the context of web development, an API such as the API layer 296 may be defined as a set of Hypertext Transfer Protocol (HTTP) request messages, along with a definition of the structure of response messages, possibly in an Extensible Markup Language (XML) or JavaScript Object Notation (JSON) format. In a Web context, APIs such as the API layer 296 may support Simple Object Access Protocol (SOAP) based web services, service-oriented architectures (SOA), direct representational state transfer (REST) style web resources, and/or resource-oriented architecture (ROA).

In various implementations, the API layer 296 is, or is included in the API layer 190 from the embodiment of FIG. 1.

In various implementations, terms such as cloud service, cloud-based service, cloud functionality, cloud-based functionality, cloud application and/or cloud-based application are used to denote software running in a computing cloud and performing various functions. Examples of such cloud-based features may include email systems, portals for accessing information stored in the cloud, applications collecting and/or analyzing data in the cloud, applications residing in the cloud and interfacing with mobile devices (e.g., mobile phones) or other user terminals, and other similar applications, features and/or services. A particularly useful class of cloud-based services are SAAS platforms providing a wide range of functionality such as sales management, data analytics and reporting, marketing management and automation, financial management and reporting, billing and payments, and other features amenable to cloud-based deployment.

As an example, data processing system 200 may be connected to cloud 290 through one or more communication channels or networks and may store data in the cloud for backup purposes and/or to enable various cloud-based services based on that data. Correspondingly, data processing system 200 may receive data from cloud 290 on demand and/or at predefined intervals. Cloud 290 may include one or more portals for administering, monitoring, configuring, and/or controlling the data processing system 200. The portal in the cloud 290 may permit one or more users to log in and access data received from the data processing system 200 and/or otherwise available in the cloud, including records of data and data analytics. In one embodiment, a cloud may perform an authentication function for a data processing system connected to the cloud, and may be configured to remotely shut down, erase, reset, update an operating system or application, or otherwise configure or restrict the operation of a remote data processing system under various circumstances (e.g., unauthorized access of the data processing system or of a cloud portal).

In various embodiments, the data processing system 200 and other systems or components shown in the embodiment of FIG. 2 (e.g., the networking device 262, the data processing system 270, the network 260, one or more of the client devices 294, the API layer 296, etc.) may be connected to a blockchain or combination of blockchains, illustrated as blockchain 298 in FIG. 2. Blockchains were discussed in more detail in connection with the embodiment of FIG. 1. In various embodiments, the blockchain 298 may represent the blockchain 196 discussed in connection with the embodiment of FIG. 1. In various embodiments, the blockchain 298 may facilitate commerce transactions and/or smart contracts involving cryptocurrencies and/or cryptographic tokens, as generally described in connection with the embodiment of FIG. 1.

FIG. 3 shows an exemplary method 300 (such method is also denoted a process, represented as process 300) for automatic translation of documents in accordance with an embodiment.

In various embodiments, the exemplary method or process for automatic translation 300 illustrated in the embodiment of FIG. 3 could be implemented on, or could run on a system for automatic translation, such as the system for automatic translation 100 discussed in connection with the embodiment of FIG. 1.

In various embodiments, the exemplary method or process for automatic translation 300 illustrated in the embodiment of FIG. 3 could be implemented on, or could run on a data processing system, such as the data processing system 102 discussed in connection with the embodiment of FIG. 1.

In various embodiments, the method or process 300 includes several steps that can process, manage and translate any number of files (including large volumes of files) automatically. In various embodiments, the method 300 is fully automatic (i.e., it does not require any human intervention). In various embodiments, the method 300 is partially automatic (i.e., one or more steps may require or may permit direct human intervention). Examples of human intervention by one or more human operators at one or more steps in the method 300 may include any of the following: (1) review and approval of a step, (2) fixing or clearing an error, or (3) any other action that may be required or appropriate. In various embodiments, the method 300 is fully or partially automatic, and permits one or more human operators to monitor the progress of the process (e.g., by displaying intermediate results, progress indicators, or other similar information to one or more human operators). For example, in various embodiments, a project manager or supervisor may monitor the evolution of the process 300. In various embodiments, a project manager or supervisor may take one or more actions, or may direct one or more other human operators to take one or more actions in response of such project manager or supervisor monitoring the evolution of the process 300.

A process that seeks to translate fast a large number of files should avoid reliance on human input in certain stages of the translation process because human input is likely to introduce delays compared to the processing speed that an automatic translation system could achieve while running on a typical high-power, high availability commercial cloud platform. Another risk with human input in a translation process is that humans could introduce errors into the translation process (e.g., in particular users with more limited training, or users who are under time pressure to complete high volumes of document translations), compared to the accuracy that an automated system can achieve if it has been properly programmed and calibrated to perform the translation process automatically.

In the embodiment of FIG. 3, the method or process 300 comprises steps 304, 306, 308, 310, 320, 330, 332, and 334 that implement various steps of the translation process. In various embodiments, one or more of the foregoing steps could be implemented through (or could run on, or could be processed by) one or more corresponding logic modules. For example, in one embodiment, each of the foregoing steps could be implemented through a corresponding logic module 104, 106, 108, 110, 120, 130, 132, and 134, which logic modules were discussed in connection with the embodiment of FIG. 1. In various embodiments, any one of the steps 304, 306, 308, 310, 320, 330, 332, and 334, or any combination of the foregoing steps could be implemented on, or could be processed by, or could run on, logic module 220 and/or logic module 223 described in connection with the embodiments of FIG. 2.

In various embodiments, each of the steps 304, 306, 308, 310, 320, 330, 332, and 334 could be implemented on, or could be processed by, or could run on, multiple logic modules, such as logic module 220 and/or logic module 223 described in connection with the embodiments of FIG. 2. For example, step 304 could be implemented on, or could be processed by, or could run on, a combination of a hardware logic module and software logic module, as described in more detail in connection with logic module 220 and logic module 223.

In various embodiments, two or more of the steps 304, 306, 308, 310, 320, 330, 332, and 334 could be combined and could be implemented on, or could be processed by, or could run on, fewer logic module. In one embodiment, a single logic module could include sufficient hardware and software to implement, process, or run all of the steps 304, 306, 308, 310, 320, 330, 332, and 334.

In various embodiments, the process or claim 300 could be implemented in, or could be processed in, or could run in, or could be deployed in a cloud. Clouds and cloud computing resources were discussed in more detail in connection with the embodiments of FIG. 2.

In various embodiments, one or more external networks, such as networks 192 and 194 discussed in connection with the embodiment of FIG. 1, provide connectivity and facilitate data transmission between one or more of the steps 304, 306, 308, 310, 320, 330, 332, and 334 and any user. In various embodiments, one or more external networks, such as networks 192 and 194 discussed in connection with the embodiment of FIG. 1, provide connectivity and facilitate data transmission between the process or method 300 and any user. In various embodiments, one or more external networks, such as networks 192 and 194 discussed in connection with the embodiment of FIG. 1, provide connectivity and facilitate data transmission between one or more of the steps 304, 306, 308, 310, 320, 330, 332, and 334 and any external data processing systems, users or entities, such as translation service providers, customers, or cloud-based storage. In various embodiments, one or more external networks, such as networks 192 and 194 discussed in connection with the embodiment of FIG. 1, provide connectivity and facilitate data transmission between the process or method 300 and any external data processing systems, users or entities, such as translation service providers, customers, or cloud-based storage.

In various embodiments, a set of original files designated for translation are accessed. One or more of the original files could be provided by a customer, or could be received or retrieved from various storage systems, databases, or other repositories. In various embodiments, at step 304, one or more of the original files may be identified, retrieved, made available, and/or prepared for pre-processing. In various embodiments, one or more of the original files are processed to ensure that such files are compatible with a format that can be handled during subsequent processing steps.

In various embodiments, the type and/or the format of any original file may be as discussed in in connection with the embodiment of FIG. 1. In various embodiments, an input supporting dataset is received at step 306. In various embodiments, the input supporting dataset may define various at attributes for the translation. This input supporting dataset may include key information about the original files to be translated, such as translation language combinations (e.g., a source language and one or more languages in which the document must be translated), document type, service type (e.g., audio, braille, translation, transcreation, document enlargement (e.g., into large print), etc.), source file name, target file name (i.e., the name that the translated document will have after translation), template identification, decryption information (e.g., a decryption key), and other metadata that assists in tailoring the translation to specific needs. The input supporting dataset can be provided by external customers, defined within system configurations, or retrieved from prior stored datasets. In various embodiments, the input supporting dataset can be used to configure the translation system to handle the specific requirements of the translation project.

In various embodiments, the input supporting dataset may be received at step 306 through an application programming interface (API), such as API lawyer 190 discussed in connection with the embodiment of FIG. 1, or through a network (e.g., network 192 or 194 discussed in connection with the embodiment of FIG. 1).

In various embodiments, once the original files and input supporting dataset are retrieved, a set of pre-processed files are automatically generated at step 308. In various embodiments, the pre-processed files are generated based on one or more of the original files. In various embodiments, the pre-processed files are generated based on the input supporting data. In various embodiments, the pre-processed files are generated based on both a set of the original files and based on the input supporting data.

In various embodiments, the automatic generation of the set of pre-processed files at step 308 can be implemented as discussed below in connection with step 410 of the embodiments of FIG. 4.

In various embodiments, the automatic generation of the set of pre-processed files at step 308 can be implemented as discussed below in connection with step 510 of the embodiments of FIG. 5.

In various embodiments, the input supporting dataset that may be received at step 306 includes metadata that corresponds to at least one of the attributes for the translation. More information regarding the input supporting dataset was discussed in connection with the logic module 106 of FIG. 1.

In various embodiments, the input supporting dataset is included in a metadata file, or is a metadata file (such metadata file that includes or consists of the input supporting dataset is denoted an “input metadata file”).

In various embodiments, the input supporting dataset is included in a companion file, or consists of a companion file, and the companion file is received directly or indirectly from a customer (e.g., received from a customer, received through a vendor of a customer, etc.). In various embodiments, the companion file defines at least one attribute for the translation. In various embodiments, the companion file is an input metadata file.

In various embodiments, the output supporting dataset includes metadata that corresponds to at least one of the translated files.

In various embodiments, the output supporting dataset is a metadata file.

In various embodiments, the output supporting dataset is based on the input supporting dataset. For example, in various embodiments, the output supporting dataset may include a portion of the input supporting dataset, or may be produced based on the input supporting dataset. In various embodiments, for example, one or more attributes that are included in the input supporting dataset are included in the output supporting dataset, or are used to produce the output supporting dataset.

In various embodiments, the metadata file that includes the output supporting dataset, or that consists of the output supporting dataset (the “output metadata file”) includes a portion of the input supporting dataset, of an input metadata file, or is otherwise based on an input supporting dataset. An input supporting dataset, input metadata file, and companion file in accordance with various embodiments are described in more detail elsewhere in this patent (e.g., in connection with the embodiment of FIG. 1 and logic module 106).

In various embodiments, the metadata file that includes the output supporting dataset, or that consists of the output supporting dataset (the “output metadata file”) includes a portion of the input supporting dataset, or is otherwise based on the input supporting dataset.

In various embodiments, the output metadata file may include a portion of the input supporting dataset, or may be produced based on the input supporting dataset. In various embodiments, for example, one or more attributes that are included in the input supporting dataset are included in the output supporting dataset, or are used to produce the output supporting dataset.

In various embodiments, the output metadata file may include a portion of an input metadata file corresponding to the input supporting dataset, or may be produced based on an input metadata file corresponding to the input supporting dataset.

In various embodiments, the output metadata file may include a portion of a companion file corresponding to the input supporting dataset, or may be produced based on a companion file corresponding to the input supporting dataset.

In various embodiments, the output metadata file may be the companion file corresponding to the input supporting dataset, or may be a variation of a companion file corresponding to the input supporting dataset.

In various embodiments, the automatic process to generate the set of pre-processed files may include one or more of the following operations:
(1) convert at least one of the original files from a format that cannot be easily edited (e.g., from Portable Document Format (PDF)) into a document format that is more easily editable (e.g., Microsoft® Word format, Google® Docs format, text format, etc.).
(2) store into a secure environment protected health information (PHI) that is included in at least one of the original files.
(3) decompress at least one of the original files.
(4) decrypt at least one of the original files.
(5) move to a different server at least one of the original files.
(6) transmit or receive through an application programming interface (API) at least one of the original files.
(7) validate at least one of the input files.
(8) create a new folder or subfolder, and place in the new folder or subfolder:
   (a) at least one of the original files, or
   (b) at least one of the pre-processed files;
(9) compare at least one of the original files with at least one template, and identify at least one portion of the original file that is different compared to the template;
(10) store at least one original file in a storage memory; and/or
(11) reproducing the original files or pre-processed files in the target language using one or more of the steps described above.

In various embodiments, the automatic generation of the set of pre-processed files takes place without any direct action from any human.

In various embodiments, one or more of the pre-processed files are subsequently made available at step 308 for translation by a logic module or by a data processing system. In various embodiments, one or more of the pre-processed files are made available at step 308 for translation at step 320.

In various embodiments, one or more of the pre-processed files generated at step 308 are subsequently made available at step 310 for translation by another logic module or by another data processing system. In various embodiments, one or more of the pre-processed files are made available at step 310 for translation at step 320.

In various embodiments, one or more of the pre-processed files may contain sensitive information, such as Protected Health Information (PHI). In such cases, a pre-processed file may be handled through a specific API that offers enhanced security. In various embodiments, this API may be different from the API that is used to make available for translation pre-processed files that do not include PHI.

In various embodiments, the pre-processed files may be made available for translation through network-based APIs, allowing remote access by translation engines or other service providers, ensuring seamless integration into the workflow. In this embodiment, pre-processed files may be stored temporarily in secure environments before translation, and access is controlled through a set of predefined protocols.

In various embodiments, one or more of the pre-processed files are translated by an AI engine at step 320. In various embodiments, translation at step 320 is implemented on, processed on, run on, or otherwise performed on a set of AI engines that translate one or more of the pre-processed files.

In various embodiments, each AI engine may be, or may include a large language model, (LLM), a neural network (NN), or any other machine learning algorithm capable of translating one or more of the pre-processed documents. AI engines were discussed in more detail in connection with the embodiments of FIG. 2.

In various embodiments, translation at step 320 is not part of the process or method 300, and instead it is performed separately. To illustrate this possibility, the border of the step 320 is shown as a dotted line in FIG. 3.

In various embodiments, translation at step 320 may be performed remotely from, in parallel with, independently from, or otherwise asynchronously with the other steps included in the process 300. In various embodiments, translation at step 320 is not part of the process 300. In various embodiments, translation at step 320 may be performed on, run on, hosted by, implemented in, or otherwise processed using a data processing system or logic module different from the data processing system or logic modules that perform, host, implement or otherwise process the steps of process 300 (e.g., translation at step 320 may be hosted by, performed or run on, implemented in, or otherwise processed in a different cloud, in a server hosted on the premises of a business entity, or in a different data processing system). In various embodiments, pre-processed files are translated at step 320. In various embodiments, one or more pre-processed files may be transmitted or made available for translation at step 320 through one or more APIs (such as the API layer 190 discussed in connection with the embodiment of FIG. 1), one or more networks (such as network 192 and/or network 194 discussed in connection with the embodiment of FIG. 1), and/or through any other communication channels.

In various embodiments, translation at step 320 may be performed remotely from, in parallel with, independently from, or otherwise asynchronously with the other steps included in the process 300. In various embodiments, one or more preprocessed files that were generated at step 308 may be transmitted or otherwise made available for translation at step 320 directly or indirectly, through one or more APIs (such as the API layer 190 discussed in connection with the embodiment of FIG. 1), one or more networks (such as network 192 and/or network 194 discussed in connection with the embodiment of FIG. 1), and/or through any other communication channels.

In various embodiments, translation at step 320 may be performed remotely from, in parallel with, independently from, or otherwise asynchronously with the other steps included in the process 300, and one or more preprocessed files that were generated by logic module 108 are translated at step 320 to produce a set of translated files. In various embodiments, one or more translated files are produced at step 320 and are made available to be accessed at step 330 and/or to be post-processed at step 332, either directly or indirectly, through one or more APIs (such as the API layer 190 discussed in connection with the embodiment of FIG. 1), one or more networks (such as network 192 and/or network 194 discussed in connection with the embodiment of FIG. 1), or through other communication channels.

In various embodiments, translation at step 320 is included in the process or method 300.

In various embodiments, a set of pre-processed files are received for translation at step 320, where one or more of those pre-preprocessed files are translated to produce a set of translated files. In various embodiments, one or more of the files translated at step 320 are subsequently made available for access at step 330.

In various embodiments, translation at step 320 is performed with, processed through, run through, or otherwise implemented using a large language model (LLM) to produce a set of translated documents. LLMs are discussed in more detail in connection with the embodiments of FIG. 2 above.

In various embodiments, an LLM uses to perform translation was trained based on a training dataset that includes translated content curated by one or more humans (e.g., training datasets that may have been curated by human translators). Such curated content may help enhance the quality and accuracy of the translations performed by process 300, especially for technical, legal, medical, or other context-sensitive documents, and for certain languages.

When deployed commercially, a system that implements various aspects of the data processing system was able to translate thousands of documents per day. More generally, a translation process similar to the process for automatic translation 300 that is properly configured and runs with adequate computing resources could be expected to translate in excess of 100, 1000, 2000, 4000, 6000, 8000, or 10000 files during a 24-hour period, and any number of files in between the foregoing figures. Moreover, if the computing resources are high enough, the volume of files that can be processed through a translation process similar to the process for automatic translation 300 described in connection with the embodiments of FIG. 3 could increase indefinitely depending on the computing resources made available to such system.

In various embodiments, one or more pre-processed files are translated at step 320 to produce a set of corresponding translated documents. In various embodiments, one or more translated documents produced through translation are made available to be accessed at step 320 and/or after step 320 for further processing.

In various embodiments, at least one of the translated files is accessed at step 330.

In various embodiments, at least one of the translated files is automatically post-processed at step 332. In various embodiments, the post-processing of the translated files may include at least one of the following:
(1) automatic quality assurance (QA) validation;
(2) moving or copying the file to a particular folder in response to an issue identified during the automatic QA validation process;
(3) changing the name of a file;
(4) review of at least one of the translated files by a human.
(5) modification of at least one of the translated files by a human;
(6) creating an automatic archive that includes at least one of the translated files;
(7) deleting at least one intermediate file that was created during the translation process (this may be helpful because the translation process could generate a significant number of intermediate files or other intermediate data, and these files or data should ideally be deleted to free up computing and memory resources);
(8) changing the name of a file (e.g., in response to a request made or rule set by a customer); and/or
(9) performing other steps to finalize a file before delivery to a client (e.g., packaging files, compressing files, encrypting files, and so on).

In various embodiments, an output supporting dataset is generated automatically at step 334 based on one or more of the translated files. In various embodiments, the output supporting dataset includes at least one of the following:
(1) invoice information related to the translation;
(2) a fee corresponding to at least one of the translated files; or
(3) reporting data for a customer based on at least one of the translated files.

In various embodiments, the automatic generation of the output supporting dataset takes place without any direct action from any human.

In various embodiments, the output supporting dataset may be transmitted to a customer or to another party (e.g., through a data processing system similar to the data processing system 200, or through a logic module such as logic module 220 or logic module 223, each of the foregoing as discussed in connection with the embodiments of FIG. 2), or may be stored in a storage memory.

In various embodiments, a network (such as network 192 or network 194 discussed in connection with the embodiment of FIG. 1) provide communication functionality for the process 300. These networks may enable the transfer of files, datasets, and communications between (a) one or more of the steps of process 300, (b) one or more logic modules that implement one or more of the steps of process 300, (c) other data processing systems, and/or (d) external entities. Network communication can occur through various protocols, including secure internet connections, private networks, or cloud-based networks, ensuring that data is transmitted efficiently and securely. These networks may also support the communication between an API layer (e.g., the API layer 190 discussed in connection with the embodiment of FIG. 1) and external components, customers, vendors, and/or storage memory resources.

In various embodiments, a set of users (such as the users 140 discussed in connection with the embodiment of FIG. 1) may perform, configure, operate, monitor, process, manage, or otherwise interact with one or more of the steps of process 300, using a personal computer, laptop, mobile phone, mobile tablet, or any other data processing system, such as the data processing systems discussed in connection with FIG. 2. In various embodiments, one or more users may have credentials that enable them to configure, monitor or otherwise use or interact with one or more of the steps of process 300.

In various embodiments, an API layer (such as the API layer 190 discussed in connection with the embodiment of FIG. 1) may facilitate data communications between one or more of the steps included in process 300, one or more users, and/or other communication endpoints.

In various embodiments, communications and file transfers between one or more of the steps of process 300 may occur via an API layer, directly via a network without using an API (e.g., using network 192 and/or network 194 illustrated in the embodiment of FIG. 1), directly via a communication channel without using an API, and/or through any combination of an API, network and communication channel. For example, in various embodiments, communications through an API layer also traverses one or more networks (e.g., a WiFi network, a cellular network, etc.). Additional discussion of networks and communication channels is provided in connection with the embodiment of FIG. 2. As another example, in various embodiments, file transfers, or communications between the one or more steps included in process 300 and a data processing system in possession of a user may take place through any of the following: (a) directly via one or more networks and without going through any APIs (e.g., a mobile app running on a consumer phone may establish an encrypted connection to an omnichannel layer and conduct a commerce transaction by transmitting and receiving data without going through an API), (b) through an API, and (c) through a combination of one or more APIs and directly via one or more networks and without going through an API (e.g., some communications may be direct and without an API, and some communications may be routed via an API).

FIG. 4 shows an exemplary method 400 (such method is also denoted a process, represented as process 400) for automatic processing of files in accordance with an embodiment.

In various embodiments, the exemplary method or process for automatic processing of files 400 illustrated in the embodiment of FIG. 4 could be implemented on, or could run on a system for automatic translation, such as the system for automatic translation 100 discussed in connection with the embodiment of FIG. 1.

In various embodiments, the exemplary method or process for automatic processing of files 400 illustrated in the embodiment of FIG. 4 could be implemented on, or could run on a data processing system, such as the data processing system 102 discussed in connection with the embodiment of FIG. 1.

In various embodiments, the method or process 400 includes several steps that can process, manage and translate any number of files (including large volumes of files) automatically. In various embodiments, the method 400 is fully automatic (i.e., it does not require any human intervention). In various embodiments, the method 400 is partially automatic (i.e., one or more steps may require or may permit direct human intervention). Examples of human intervention by one or more human operators at one or more steps in the method 400 may include any of the following: (1) review and approval of a step, (2) fixing or clearing an error, or (3) any other action that may be required or appropriate. In various embodiments, the method 400 is fully or partially automatic, and permits one or more human operators to monitor the progress of the process (e.g., by displaying intermediate results, progress indicators, or other similar information to one or more human operators). For example, in various embodiments, a project manager or supervisor may monitor the evolution of the process 400. In various embodiments, a project manager or supervisor may take one or more actions, or may direct one or more other human operators to take one or more actions in response of such project manager or supervisor monitoring the evolution of the process 400.

In the embodiment of FIG. 4, the method or process 400 comprises steps 404, 406, 408, 410 and 420 that implement various steps of the automatic processing of files. In various embodiments, one or more of the foregoing steps could be implemented through (or could run on, or could be processed by) one or more corresponding logic modules. For example, in one embodiment, each of the foregoing steps could be implemented through a corresponding logic module, as discussed in connection with the embodiments of FIG. 1 or FIG. 2. In various embodiments, any one of the steps 404, 406, 408, 410 and 420, or any combination of the foregoing steps could be implemented on, or could be processed by, or could run on, logic module 220 and/or logic module 223 described in connection with the embodiments of FIG. 2.

In various embodiments, each of the steps 404, 406, 408, 410 and 420 could be implemented on, or could be processed by, or could run on, multiple logic modules, such as logic module 220 and/or logic module 223 described in connection with the embodiments of FIG. 2. For example, step 404 could be implemented on, or could be processed by, or could run on, a combination of a hardware logic module and software logic module, as described in more detail in connection with logic module 220 and logic module 223.

In various embodiments, two or more of the steps 404, 406, 408, 410 and 420 could be combined and could be implemented on, or could be processed by, or could run on, fewer logic module. In one embodiment, a single logic module could include sufficient hardware and software to implement, process, or run all of the steps 404, 406, 408, 410 and 420.

In various embodiments, the process or claim 400 could be implemented in, or could be processed in, or could run in, or could be deployed in a cloud. Clouds and cloud computing resources were discussed in more detail in connection with the embodiments of FIG. 2.

In various embodiments, one or more external networks, such as networks 192 and 194 discussed in connection with the embodiment of FIG. 1, provide connectivity and facilitate data transmission between one or more of the steps 404, 406, 408, 410 and 420 and any user. In various embodiments, one or more external networks, such as networks 192 and 194 discussed in connection with the embodiment of FIG. 1, provide connectivity and facilitate data transmission between the process or method 400 and any user. In various embodiments, one or more external networks, such as networks 192 and 194 discussed in connection with the embodiment of FIG. 1, provide connectivity and facilitate data transmission between one or more of the steps 404, 406, 408, 410 and 420 and any external data processing systems, users or entities, such as translation service providers, customers, or cloud-based storage. In various embodiments, one or more external networks, such as networks 192 and 194 discussed in connection with the embodiment of FIG. 1, provide connectivity and facilitate data transmission between the process or method 400 and any external data processing systems, users or entities, such as translation service providers, customers, or cloud-based storage.

In various embodiments, a set of original files designated for processing (e.g. format conversion, translation, etc.) are accessed at step 404. One or more of the original files could be provided by a customer, or could be received or retrieved from various storage systems, databases, or other repositories. In various embodiments, at step 404, one or more of the original files may be identified, retrieved, made available, and/or prepared for pre-processing. In various embodiments, one or more of the original files are processed to ensure that such files are compatible with a format that can be handled during subsequent processing steps.

In various embodiments, the type and/or the format of any original file may be as discussed in in connection with the embodiment of FIG. 1.

In various embodiments, a set of templates is accessed at step 406. This set of templates may include one or more templates. In some embodiments, one or more of these templates are produced based on one or more of the original files accessed at step 404. In some embodiments, one or more of these templates are received or retrieved from a source (e.g., provided by a customer, retrieved from a memory system, retrieved or received through an API, etc.).

In various embodiments, a template may represent a particular type of file or a particular structure of file, in whole or in part. In various embodiments, a template may represent a legal document (e.g., a legal agreement, a legal form, a legal application to a court or to a governmental agency, etc.), an application for a benefit (e.g., an application for a governmental agency (e.g., an application for a driver's license or for an immigration visa, an application for a permit, an application for employment, etc.), an application to a private company (e.g., an application for insurance (e.g., vehicle insurance, home insurance, medical insurance, etc.), an application for employment, etc.), a business document (e.g., a business report, etc.), an accounting or financial document (e.g., a financial report, an accounting statement, etc.), a letter (e.g., a letter from a governmental agency, a letter from an insurance provider (e.g., vehicle insurance, home insurance, medical insurance, etc.), etc.), a letter from a financial institution, a denial letter (e.g., a letter denying an application or a benefit), etc.), an advertisement, an email or other written or electronic communication sent to multiple recipients, a customer response letter, a benefits statement (e.g., from a medical provider, governmental entity, insurance company, etc.), and so on. In various embodiments, hundreds of templates may exist, and one or more of such templates may be available to be accessed at step 406.

In various embodiments, at least one of the templates accessed at step 406 is produced (or generated) based on one or more of the original files accessed at step 404. In various embodiments, to produce a template, two or more original files may be processed (e.g., the files may be compared, the content of the files may be run through a correlation algorithm or through some other process known in the art to determine similarity of content, etc.). In various embodiments, as a result of such processing, certain portions of such files may be determined to be identical or similar, and such identical or similar portions of the files maty be used to produce a template. In various embodiments, the processing of the two or more original files, and/or the determination that certain portions are identical or similar, may be performed by a set of logic modules, a set of data processing systems, or a combination of the foregoing.

In various embodiments, at least one of the templates accessed at step 406 is received or retrieved from a source (e.g., provided by a customer, retrieved from a memory system, retrieved or received through an API, etc.). In various embodiments, at least one of the templates accessed at step 406 is selected based on the content of one or more original files accessed at step 404 (e.g., the content of an original file may be determined to correspond to a particular template, and such template (or a similar template) may be accessed at step 406).

In various embodiments, a template may be pre-processed in whole or in part (e.g., partially or fully translated).

In the embodiment of FIG. 4, certain variable content is determined at step 408. In various embodiments, such variable content may be data that is populated in a template. For example, in various embodiments, a template may be a letter that an insurance company sends to a customer, and the variable content may be the name of the customer, the address of the customer, a customer or account number, and other information that relates to such customer or that relates to the substance of the communication directed to such customer. In various embodiments, the variable content may represent information that tends to change among recipients of documents that are based on the same template.

In the embodiment of FIG. 4, a set of pre-processed files are generated automatically at step 410 based on at least a portion of the variable content determined at step 408 and based one or more of the templates accessed at step 408.

In various embodiments, the automatic process to produce (or generate) the set of pre-processed files at step 410 may include one or more of the following operations:
(1) convert at least one of the original files from a format that cannot be easily edited (e.g., from Portable Document Format (PDF)) into a document format that is more easily editable (e.g., Microsoft® Word format, Google® Docs format, text format, etc.).
(2) store into a secure environment protected health information (PHI) that is included in at least one of the original files.
(3) decompress at least one of the original files.
(4) decrypt at least one of the original files.
(5) move to a different server at least one of the original files.
(6) transmit or receive through an application programming interface (API) at least one of the original files.
(7) validate at least one of the input files.
(8) create a new folder or subfolder, and place in the new folder or subfolder:
   (a) at least one of the original files, or
   (b) at least one of the pre-processed files;
(9) compare at least one of the original files with at least one template, and identify at least one portion of the original file that is different compared to the template;
(10) store at least one original file in a storage memory; and/or
(11) reproducing the original files or pre-processed files in the target language using one or more of the steps described above.

In various embodiments, the automatic generation of the set of pre-processed files takes place without any direct action from any human.

In various embodiments, the automatic processing of the original files may include translation of at least a portion of at least one of the original files. In the embodiment of FIG. 4, the determination of variable content shown at step 408 is connected a dotted line to the translation shown at step 422 to illustrate that in various embodiments, the translation at step 422 may follow the determination of the variable content at step 408, and therefore the translation could be performed as part of the automatic production (or generation) of the set of pre-processed files at step 410, or in parallel with the automatic production (or generation) of the set of pre-processed files at step 410.

In various embodiments, the automatic processing of the original files may be followed by translation of at least a portion of at least one of the original files. In the embodiment of FIG. 4, the translation of one or more pre-processed files shown at step 420 is connected by dotted lines to step 408, where certain variable content was determined, to illustrate that translation may follow the determination of the variable content at step 408, and therefore the translation could be performed based on the pre-processed files that were automatically generated at step 410.

In some embodiments, translation may occur both at step 420 based on the pre-processed files that were automatically generated at step 410, and at step 422 based on the variable content determined at step 408. In some embodiments, such translation at step 420 and at step 422 may apply to different original files that were accessed at step 404. In some embodiments, such translation at step 420 and at step 422 may apply to a particular original file that was accessed at step 404 (e.g., such translation may use two different approaches and may be used to check the accuracy of either of the two approaches, or to improve the overall translation of the original file).

In various embodiments, the translation at step 420 and/or at step 422 may be based on certain variable content determined at step 408 and on a template accessed at step 406 as follows: (1) translate the variable content, (2) retrieve a translated version of the template (e.g., from a memory system, through an API, etc.), and (3) assemble a post-processed file (i.e., a translated file) by integrating the translated variable content into the translated version of the template.

In various embodiments, the translation at step 420 and/or at step 422 may be based on certain variable content determined at step 408 and on a template accessed at step 406 as follows: (1) translate the variable content, (2) translate the template, and (3) assemble a post-processed file (i.e., a translated file) by integrating the translated variable content into the translated template.

For example, in one embodiment, an original file may be a letter from an insurance company directed to a customer, and such letter may be designated for translation from a source language into a target language. In one embodiment, the original file may be accessed at step 404, a template corresponding to such letter may be retrieved or produced at step 406, certain variable content may be determined at step 408 (e.g., the name and account number of the customer), and the original file may be processed automatically at step 410 to generate a set of pre-processed files through one or more of the operations described above in connection with step 410. In various embodiments, the automatic processing at step 410 may include translation of at least a portion of the original file at step 422. In various embodiments, the automatic processing at step 410 may be followed by translation at step 420 of at least a portion of at least one pre-processed file generated at step 410.

In various embodiments, step 410 where one or more original files are processed automatically to generate a set of pre-processed files corresponds to, is the same as, or is equivalent to step 308 from the embodiment of FIG. 3. In various embodiments, one or more of the pre-processed files that are generated automatically at step 410 may be made available for translation at step 310 of the embodiment of FIG. 3.

In various embodiments, one or more of the pre-processed files that are automatically generated at step 410 may be one or more of the pre-processed files that are made available for translation at step 310 in the embodiment of FIG. 3.

In various embodiments, step 420 where one or more pre-processed files are translated corresponds to, is the same as, or is equivalent to step 320 from the embodiment of FIG. 3. In various embodiments, one or more of the pre-processed files that are translated at step 420 may be accessed in step 330 of the embodiment of FIG. 3.

In various embodiments, an input supporting dataset is received in connection with the process described for the embodiment of FIG. 4. In various embodiments, such input supporting dataset is the input supporting dataset from step 306 of FIG. 3. In various embodiments, such input supporting dataset may define various attributes for the translation. Various exemplary attributes and content of such input supporting dataset were discussed in connection with step 306 of FIG. 3.

In various embodiments, such input supporting dataset may also include information that identifies a particular set of templates to be accessed at step 406. For example, such input supporting dataset may identify a particular template as an insurance letter, a business document, or a legal document. In various embodiments, one or more of the templates accessed at step 406 are selected based on information in such input supporting dataset.

In various embodiments, such input supporting dataset includes metadata that corresponds to at least one of the attributes for the translation. In various embodiments, such input supporting dataset is a metadata file. In various embodiments, such input supporting dataset is a companion file received directly or indirectly from a customer (e.g., received from a customer, received through a vendor of a customer, etc.), and the companion file defines at least one attribute for the translation and includes information that can be used to select one or more templates accessed at step 406.

In various embodiments, one or more of the templates accessed at step 406 are produced or are selected using a set of AI engines that process one or more of the original files accessed at step 404 and/or at least a portion of the input supporting dataset. In various embodiments, each AI engine may be, or may include a large language model, (LLM), a neural network (NN), or any other machine learning algorithm capable of translating one or more of the pre-processed documents. AI engines were discussed in more detail in connection with the embodiments of FIG. 2.

FIG. 5 shows an exemplary method 500 (such method is also denoted a process, represented as process 500) for automatic processing of files in accordance with an embodiment.

In various embodiments, the exemplary method or process for automatic processing of files 500 illustrated in the embodiment of FIG. 5 could be implemented on, or could run on a system for automatic translation, such as the system for automatic translation 100 discussed in connection with the embodiment of FIG. 1. In various embodiments, the exemplary method or process for automatic processing of files 500 illustrated in the embodiment of FIG. 5 could be implemented on, or could run on a data processing system, such as the data processing system 102 discussed in connection with the embodiment of FIG. 1.

In various embodiments, the method or process 500 includes several steps that can process, manage and translate any number of files (including large volumes of files) automatically. In various embodiments, the method 500 is fully automatic (i.e., it does not require any human intervention). In various embodiments, the method 500 is partially automatic (i.e., one or more steps may require or may permit direct human intervention). Examples of human intervention by one or more human operators at one or more steps in the method 500 may include any of the following: (1) review and approval of a step, (2) fixing or clearing an error, or (3) any other action that may be required or appropriate. In various embodiments, the method 300 is fully or partially automatic, and permits one or more human operators to monitor the progress of the process (e.g., by displaying intermediate results, progress indicators, or other similar information to one or more human operators). For example, in various embodiments, a project manager or supervisor may monitor the evolution of the process 500. In various embodiments, a project manager or supervisor may take one or more actions, or may direct one or more other human operators to take one or more actions in response of such project manager or supervisor monitoring the evolution of the process 500..

In the embodiment of FIG. 5, the method or process 500 comprises steps 504, 508, 510 and 520 that implement various steps of the automatic processing of files. In various embodiments, one or more of the foregoing steps could be implemented through (or could run on, or could be processed by) one or more corresponding logic modules. For example, in one embodiment, each of the foregoing steps could be implemented through a corresponding logic module, as discussed in connection with the embodiments of FIG. 1 or FIG. 2. In various embodiments, any one of the steps 504, 508, 510 and 520, or any combination of the foregoing steps could be implemented on, or could be processed by, or could run on, logic module 220 and/or logic module 223 described in connection with the embodiments of FIG. 2.

In various embodiments, each of the steps 504, 508, 510 and 520 could be implemented on, or could be processed by, or could run on, multiple logic modules, such as logic module 220 and/or logic module 223 described in connection with the embodiments of FIG. 2. For example, step 504 could be implemented on, or could be processed by, or could run on, a combination of a hardware logic module and software logic module, as described in more detail in connection with logic module 220 and logic module 223.

In various embodiments, two or more of the steps 504, 508, 510 and 520 could be combined and could be implemented on, or could be processed by, or could run on, fewer logic module. In one embodiment, a single logic module could include sufficient hardware and software to implement, process, or run all of the steps 504, 508, 510 and 520.

In various embodiments, the process or claim 500 could be implemented in, or could be processed in, or could run in, or could be deployed in a cloud. Clouds and cloud computing resources were discussed in more detail in connection with the embodiments of FIG. 2.

In various embodiments, one or more external networks, such as networks 192 and 194 discussed in connection with the embodiment of FIG. 1, provide connectivity and facilitate data transmission between one or more of the steps 504, 508, 510 and 520 and any user. In various embodiments, one or more external networks, such as networks 192 and 194 discussed in connection with the embodiment of FIG. 1, provide connectivity and facilitate data transmission between the process or method 500 and any user. In various embodiments, one or more external networks, such as networks 192 and 194 discussed in connection with the embodiment of FIG. 1, provide connectivity and facilitate data transmission between one or more of the steps 504, 508, 510 and 520 and any external data processing systems, users or entities, such as translation service providers, customers, or cloud-based storage. In various embodiments, one or more external networks, such as networks 192 and 194 discussed in connection with the embodiment of FIG. 1, provide connectivity and facilitate data transmission between the process or method 500 and any external data processing systems, users or entities, such as translation service providers, customers, or cloud-based storage.

In various embodiments, the process illustrated in FIG. 5 is similar to the process that was discussed in connection with the embodiments of FIG. 4, except that step 406 from the embodiments of FIG. 4, where a set of templates were accessed, does not have a corresponding step in the process illustrated in FIG. 5. In various embodiments, no template is used to identify content to be processed at step 508 of the embodiments of FIG. 5. In various embodiments, all content of one or more original files accessed at step 504 is considered variable content.

In various embodiments, a set of original files designated for processing (e.g. format conversion, translation, etc.) are accessed at step 504. One or more of the original files could be provided by a customer, or could be received or retrieved from various storage systems, databases, or other repositories. In various embodiments, at step 504, one or more of the original files may be identified, retrieved, made available, and/or prepared for pre-processing. In various embodiments, one or more of the original files are processed to ensure that such files are compatible with a format that can be handled during subsequent processing steps.

In various embodiments, the type and/or the format of any original file may be as discussed in in connection with the embodiment of FIG. 1.

In the embodiment of FIG. 4, certain content is identified for processing at step 508. In various embodiments, such content is identified to be processed based the nature of the content itself (e.g., if the content is determined to relate to, or resemble the name of a customer, the address of a customer, an account number, etc.). In various embodiments, all content of one or more of the original files accessed at step 504 is identified for processing (e.g., all content may be considered variable content, as discussed in connection with the embodiments of FIG. 4).

In the embodiment of FIG. 5, a set of pre-processed files are generated automatically at step 510 based on at least a portion of the content identified for processing at step 508.

In various embodiments, the automatic process to produce (or generate) the set of pre-processed files at step 510 may include one or more of the following operations:
(1) convert at least one of the original files from a format that cannot be easily edited (e.g., from Portable Document Format (PDF)) into a document format that is more easily editable (e.g., Microsoft® Word format, Google® Docs format, text format, etc.).
(2) store into a secure environment protected health information (PHI) that is included in at least one of the original files.
(3) decompress at least one of the original files.
(4) decrypt at least one of the original files.
(5) move to a different server at least one of the original files.
(6) transmit or receive through an application programming interface (API) at least one of the original files.
(7) validate at least one of the input files.
(8) create a new folder or subfolder, and place in the new folder or subfolder:
   (a) at least one of the original files, or
   (b) at least one of the pre-processed files;
(9) compare at least one of the original files with at least one template, and identify at least one portion of the original file that is different compared to the template;
(10) store at least one original file in a storage memory; and/or
(11) reproducing the original files or pre-processed files in the target language using one or more of the steps described above.

In various embodiments, the automatic generation of the set of pre-processed files takes place without any direct action from any human.

In various embodiments, the automatic processing of the original files may include translation of at least a portion of at least one of the original files. In the embodiment of FIG. 5, the determination of content to be processed at step 508 is connected a dotted line to the translation shown at step 522 to illustrate that in various embodiments, the translation at step 522 may follow the determination of the content to be processed at step 508, and therefore the translation could be performed as part of the automatic production (or generation) of the set of pre-processed files at step 510, or in parallel with the automatic production (or generation) of the set of pre-processed files at step 510.

In various embodiments, the automatic processing of the original files may be followed by translation of at least a portion of at least one of the original files. In the embodiment of FIG. 5, the translation of one or more pre-processed files shown at step 520 is connected by dotted lines to step 508, where certain content was identified for processing, to illustrate that translation may follow the determination of the content to be processed at step 508, and therefore the translation could be performed based on the pre-processed files that were automatically generated at step 510.

In some embodiments, translation may occur both at step 520 based on the pre-processed files that were automatically generated at step 510, and at step 522 based on the content that was identified for processing at step 508. In some embodiments, such translation at step 520 and at step 522 may apply to different original files that were accessed at step 504. In some embodiments, such translation at step 520 and at step 522 may apply to a particular original file that was accessed at step 504 (e.g., such translation may use two different approaches and may be used to check the accuracy of either of the two approaches, or to improve the overall translation of the original file).

In various embodiments, the translation at step 520 and/or at step 522 may be based on certain content that was identified for processing at step 508 as follows: (1) translate at least a portion of the content identified for processing, and (2) produce (or generate) a post-processed file (i.e., a translated file) based on the translated content.

For example, in one embodiment, an original file may be a letter from an insurance company directed to a customer, and such letter may be designated for translation from a source language into a target language. In one embodiment, the original file may be accessed at step 504, certain content from the original file may be identified for processing at step 508 (e.g., the name and account number of the customer, other text included in the original file, etc.), and the original file may be processed automatically at step 510 to generate a set of pre-processed files through one or more of the operations described above in connection with step 510. In various embodiments, the automatic processing at step 510 may include translation of at least a portion of the original file at step 522. In various embodiments, the automatic processing at step 510 may be followed by translation at step 520 of at least a portion of at least one pre-processed file generated at step 510.

In various embodiments, step 510 where one or more original files are processed automatically to generate a set of pre-processed files corresponds to, is the same as, or is equivalent to step 308 from the embodiment of FIG. 3. In various embodiments, one or more of the pre-processed files that are generated automatically at step 510 may be made available for translation at step 310 of the embodiment of FIG. 3.

In various embodiments, one or more of the pre-processed files that are automatically generated at step 510 may be one or more of the pre-processed files that are made available for translation at step 310 in the embodiment of FIG. 3.

In various embodiments, step 520 where one or more pre-processed files are translated corresponds to, is the same as, or is equivalent to step 320 from the embodiment of FIG. 3. In various embodiments, one or more of the pre-processed files that are translated at step 520 may be accessed in step 330 of the embodiment of FIG. 3.

In various embodiments, an input supporting dataset is received in connection with the process described for the embodiment of FIG. 5. In various embodiments, such input supporting dataset is the input supporting dataset from step 306 of FIG. 3. In various embodiments, such input supporting dataset may define various attributes for the translation. Various exemplary attributes and content of such input supporting dataset were discussed in connection with step 306 of FIG. 3.

In various embodiments, such input supporting dataset may also include information that identifies content to be processed. For example, such input supporting dataset may identify a particular portion of an input file (e.g., an insurance letter, a business document, or a legal document) to be processed (e.g., to be converted into a different format, to be translated into a different language, etc.). In various embodiments, some or all of the content identified for processing at step 508 is identified based on information in such input supporting dataset.

In various embodiments, such input supporting dataset includes metadata that corresponds to at least one of the attributes for the translation. In various embodiments, such input supporting dataset is a metadata file. In various embodiments, such input supporting dataset is a companion file received directly or indirectly from a customer (e.g., received from a customer, received through a vendor of a customer, etc.), and the companion file defines at least one attribute for the translation and includes information that can be used to identify content for processing at step 508.

In various embodiments, some or all of the content identified for processing at step 508 is identified using a set of AI engines that process one or more of the original files accessed at step 504 and/or at least a portion of the input supporting dataset. In various embodiments, each AI engine may be, or may include a large language model, (LLM), a neural network (NN), or any other machine learning algorithm capable of translating one or more of the pre-processed documents. AI engines were discussed in more detail in connection with the embodiments of FIG. 2.

Some of the embodiments described in this application (or, upon issuance, patent) may be presented in terms of algorithms and symbolic representations of operations on data bits within a computer memory. In general, an algorithm represents a sequence of steps leading to a desired result. Such steps generally require physical manipulations of physical quantities. Usually, though not necessarily, these quantities take the form of electrical or magnetic signals capable of being stored, transferred, combined, compared, and otherwise manipulated using appropriate electronic devices. Such signals may be denoted as bits, values, elements, symbols, characters, terms, numbers, or using other similar terminology.

When used in connection with the manipulation of electronic data, terms such as processing, computing, calculating, determining, displaying, or the like, refer to the action and processes of a computer system or other electronic system that manipulates and transforms data represented as physical (electronic) quantities within the system's registers and memories into other data similarly represented as physical quantities within the memories or registers of that system of or other information storage, transmission or display devices.

Various embodiments of the present invention may be implemented using an apparatus or machine that executes programming instructions. Such an apparatus or machine may be specially constructed for the required purposes, or may comprise a general purpose computer selectively activated or reconfigured by a software application.

Algorithms discussed in connection with various embodiments are not inherently related to any particular computer or other apparatus. Various general purpose systems may be used with programs in various embodiments, or in some embodiments more specialized systems, devices or components could be deployed to perform the respective functions. Embodiments are not described with reference to any particular programming language, data transmission protocol, or data storage protocol. Instead, a variety of programming languages, transmission or storage protocols may be used to implement various embodiments.

This specification describes in detail various embodiments and implementations of the present invention, and the present invention is open to additional embodiments and implementations, further modifications, and alternative and/or complementary constructions. There is no intention in this patent to limit the invention to the particular embodiments and implementations disclosed; on the contrary, this patent is intended to cover all modifications, equivalents and alternative embodiments and implementations that fall within the scope of the claims.

As used in this specification, a set means any group of one, two or more items. Analogously, a subset means, with respect to a set of N items, any group of such items consisting of N-1 or less of the respective N items.

In general, unless otherwise stated or required by the context, when used in this patent application (or, upon issuance, in this patent) in connection with a method or process, data processing system, or logic module, the words “adapted” and “configured” are intended to describe that the respective method, data processing system or logic module is capable of performing the respective functions by being appropriately adapted or configured (e.g., via programming, via the addition of relevant components or interfaces, etc.), but are not intended to suggest that the respective method, data processing system or logic module is not capable of performing other functions. For example, unless otherwise expressly stated, a logic module that is described as being adapted to process a specific class of information will not be construed to be exclusively adapted to process only that specific class of information, but may in fact be able to process other classes of information and to perform additional functions (e.g., receiving, transmitting, converting, or otherwise processing or manipulating information).

As used in this patent application (or, upon issuance, in this patent), the terms “include,” “including,” “for example,” “exemplary,” “e.g.,” and variations thereof, are not intended to be terms of limitation, but rather are intended to be followed by the words “without limitation” or by words with a similar meaning. Definitions in this specification, and all headers, titles and subtitles, are intended to be descriptive and illustrative with the goal of facilitating comprehension, but are not intended to be limiting with respect to the scope of the inventions as recited in the claims. Each such definition is intended to also capture additional equivalent items, technologies or terms that would be known or would become known to a person of average skill in this art as equivalent or otherwise interchangeable with the respective item, technology or term so defined. Unless otherwise required by the context, the verb “may” or “could” indicates a possibility that the respective action, step or implementation may or could be achieved, but is not intended to establish a requirement that such action, step or implementation must occur, or that the respective action, step or implementation must be achieved in the exact manner described.

As used in this specification, when applied to a set of two or more terms (e.g., a list of two or more words, elements, steps, systems, processes, components, or other items of any kind), the phrase “and/or” is intended to capture every possible combination of such terms to the extent applicable in the context, including each term alone, every combination of such terms, and all terms together. For example, “A, B and/or C” is intended to mean each of the following to the extent applicable in the context: (1) A, or (2) B, or (3) C, or (4) A and B, or (5) A and C, or (6) A, B and C.

As used in this specification, when applied to a set of two or more terms (e.g., a list of two or more words, elements, steps, systems, processes, components, or other items of any kind), the phrase “at least one of the following” is intended to capture every possible combination of such terms to the extent applicable in the context, including each term alone, every combination of such terms, and all terms together. For example, “at least one of the following: A, B or C” is intended to mean each of the following to the extent applicable in the context: (1) A, or (2) B, or (3) C, or (4) A and B, or (5) A and C, or (6) A, B and C.

What is claimed is:

1. A data processing system for automatic preprocessing of files for translation, the data processing system comprising a set of logic modules that are configured to:
access a set of original files to be translated;
receive a set of attributes for the translation;
generate automatically a set of pre-processed files based on at least one of the original files and based on at least one of the attributes; and
make available at least one pre-processed file for translation.

2. The data processing system of claim 1, wherein the automatic generation of the set of pre-processed files takes place without any direct action from any human.

3. The data processing system of claim 1, wherein the attributes are included in an input supporting dataset.

4. The data processing system of claim 3, wherein at least one of the following applies:
the input supporting dataset includes metadata that corresponds to at least one of the attributes for the translation; or
the input supporting dataset is included in, or is a metadata file.

5. The data processing system of claim 4, wherein the input supporting dataset is included in, or is a companion file received directly or indirectly from a customer, and wherein the companion file defines at least one of the attributes for the translation.

6. The data processing system of claim 5, wherein the companion file is received from a remote online system, via email, or through an application programming interface (API).

7. The data processing system of claim 1, wherein the set of attributes for the translation includes at least one of the following:
document type;
ID for a template type;
translation language combination;
service type;
source file name; or
target file name.

8. The data processing system of claim 7, wherein the service type includes at least one of the following:
audio;
braille;
translation;
transcreation;
conversion to large print;
conversation to standard print; or
document enlargement.

9. The data processing system of claim 1, wherein the output supporting dataset includes information indicating whether the translation process was successful.

10. The data processing system of claim 1, wherein at least one of the translated files is translated using an AI engine.

11. The data processing system of claim 10, wherein the AI engine is one of the following:
a large language model (LLM);
a neural network (NN) that is not an LLM; or a machine learning algorithm that is not an LLM or a NN.

12. The data processing system of claim 10, wherein the AI engine was trained based on a training
dataset that includes translated content curated by one or more humans

13. The data processing system of claim 1, wherein the number of translated files exceeds one or more of the following during any period of twenty-four consecutive hours:
100;
1000;
2000;
4000;
6000;
8000; or
10000.

14. The data processing system of claim 1, wherein the automatic generation of the set of pre-processed files includes at least one of the following:
convert at least one of the original files from Portable Document Format (PDF) into a document format that is editable with non-PDF document processing software;
store into a secure environment protected health information (PHI) that is included in at least one of the original files;
decompress at least one of the original files;
decrypt at least one of the original files;
move to a different server at least one of the original files;
transmit or receive through an application programming interface (API) at least one of the original files;
validate at least one of the input files;
create a new folder or subfolder, and place in the new folder or subfolder:
at least one of the original files; or
at least one of the pre-processed files;
compare at least one of the original files with at least one template, and identify at least one portion of the original file that is different compared to the template;
compare at least one of the original files with content that was previously translated, and identify at least one portion of the original file that is different compared to the content that was previously translated; or
store at least one original file in a storage memory.

15. The data processing system of claim 1, wherein at least one pre-processed file includes protected health information (PHI), and wherein the at least one pre-processed file is made available for translation through an application programming interface (API) that is different from the API used to make available for translation pre-processed files that do not include PHI.

16. A method for automatic processing of files for translation, wherein the method is implemented on a data processing system comprising a set of logic modules that are configured to perform the following steps:
access a set of original files to be translated;
receive a set of attributes for the translation;
generate automatically a set of pre-processed files based on at least one of the original files and based on at least one of the attributes; and
make available at least one of the pre-processed file for translation.

17. The method of claim 16, wherein the automatic generation of the set of pre-processed files takes place without any direct action from any human.

18. The method of claim 16, wherein the automatic generation of the set of pre-processed files includes at least one of the following:
convert at least one of the original files from Portable Document Format (PDF) into a document format that is editable with non-PDF document processing software;
store into a secure environment protected health information (PHI) that is included in at least one of the original files;
decompress at least one of the original files;
decrypt at least one of the original files;
move to a different server at least one of the original files;
transmit or receive through an application programming interface (API) at least one of the original files;
validate at least one of the input files;
create a new folder or subfolder, and place in the new folder or subfolder:
at least one of the original files; or
at least one of the pre-processed files;
compare at least one of the original files with at least one template, and identify at least one portion of the original file that is different compared to the template;
compare at least one of the original files with content that was previously translated, and identify at least one portion of the original file that is different compared to the content that was previously translated; or
store at least one original file in a storage memory.

19. The method of claim 16, wherein at least one pre-processed file includes protected health information (PHI), and wherein the at least one pre-processed file is made available for translation through an application programming interface (API) that is different from the API used to make available for translation pre-processed files that do not include PHI.
